(19) United States
(12) Patent Application Publication
(10) Pub. No.: US 2025/0186443 A1
(43) Pub. Date: Jun. 12, 2025

(54) VALACYCLOVIR AND CELECOXIB IN COMBINATION WITH NIRMATRELVIR AND RITONAVIR FOR THE TREATMENT OF COVID-19

(71) Applicant: William Langley PRIDGEN, Tuscaloosa, AL (US)

(72) Inventor: William Langley PRIDGEN, Tuscaloosa, AL (US)

(21) Appl. No.: 18/971,251

(22) Filed: Dec. 6, 2024

Related U.S. Application Data
(60) Provisional application No. 63/607,318, filed on Dec. 7, 2023.

(51) Int. Cl. A61K 31/513; A61K 31/403; A61K 31/415; A61K 31/427; A61K 31/52; A61P 31/14

(52) U.S. Cl. CPC A61K 31/513; A61K 31/403; A61K 31/415; A61K 31/427; A61K 31/52; A61P 31/14

(57) ABSTRACT

The present disclosure relates to methods of diseases and/or conditions associated with Covid-19 infection, including long COVID, comprising administration of a COX-2 inhibitor, an antiviral compound, and one or more additional active ingredients, such as a combination of nirmatrelvir and ritonavir, molnupiravir, BCG vaccine, or ivermectin.

### CROSS-REFERENCE TO RELATED APPLICATIONS

This application claims priority to U.S. Ser. No. 63/607,318, filed on 7 Dec. 2023. The entire disclosure of the application identified in this paragraph is incorporated herein by reference.

### FIELD

The present disclosure relates to methods of treating human diseases including treating or ameliorating diseases or conditions associated with SARS-COV2 infections and/or treating or ameliorating a post-acute infection syndrome.

### BACKGROUND

Infection with SARS-COV2 was first reported around November 2019. Since then, the virus has spread across the world causing an illness generally referred to as “COVID-19.” Estimates suggest that between 14% and 19% of people with COVID-19 develop long-term symptoms. [Logue, et al., J. Am. Med. Assoc. (2020); Garrigues et al., J. Infect. 81(6):e4-e6 (2020).] The chronic condition is commonly known as “long COVID,” “long haul COVID,” long-hauler's disease,” or post-acute sequelae of SARS COV-2″ (PASC)”. Evidence suggests that long COVID is significantly underreported. There are currently no established, FDA-approved treatments for long COVID.

Post-acute infection syndrome (PAIS), which includes PASC, can be a sequelae of infection with a virus or bacteria. PAIS can lead to severe chronic fatigue, exercise intolerance, autonomic dysfunction, unrefreshing sleep, memory impairment, and mood changes. See Choutka, et al. (Nat Med, May 2022; 28:911-923). In some cases, PAIS can lead to complex multisystem disorders, such as ME/CFS, fibromyalgia, and irritable bowel syndrome. See Komaroff, et al. (Front Med, January 2021; 7).

U.S. Pat. No. 8,809,351 describes various combinations of an antiviral compound (e.g., famciclovir or valacyclovir) and a COX-2 inhibitor (e.g., celecoxib) for the treatment of functional somatic syndromes (FSS) and fibromyalgia.

Although effective for acute COVID-19, PAXLOVID™ (a combination of nirmatrelvir with ritonavir) has not shown any efficacy for treating long-COVID. The STOP-PASC clinical trial (NCT05576662) was initiated to explore treatment of long-COVID with PAXLOVID. However, the clinical trial ended early, and enrollment was halted following inconclusive evidence for the primary outcome of treating long-COVID.

### SUMMARY

In one embodiment, there is provided a synergistic composition comprising a COX-2 inhibitor, an antiviral compound, nirmatrelvir, and ritonavir.

In one embodiment, there is provided a method for treating a subject susceptible to or afflicted with COVID-19, including long COVID-19, the method comprising administering to the subject a therapeutically-effective combination of a COX-2 inhibitor, an antiviral compound, and one or more additional active ingredients, such as a combination of nirmatrelvir and ritonavir, molnupiravir, Bacille Calmette-Guerin (BCG) vaccine, and/or ivermectin.

In one embodiment, there is provided a method for treating symptoms of long COVID-19 a subject susceptible to or afflicted with long COVID-19, the method comprising administering to the subject a therapeutically-effective combination of a COX-2 inhibitor, an antiviral compound, and one or more additional active ingredients.

In one embodiment, there is provided a method for treating a subject susceptible to or afflicted with PAIS, the method comprising administering to the subject a therapeutically-effective combination of a COX-2 inhibitor, an antiviral compound, and one or more additional active ingredients.

In one embodiment, there is provided a method for treating a symptoms of orthostatic intolerance in a subject suffering from PAIS, the method comprising administering to the subject a therapeutically-effective combination of a COX-2 inhibitor, an antiviral compound, and one or more additional active ingredients.

Further areas of applicability will become apparent from the description provided herein. The description and specific examples in this summary are intended for purposes of illustration only and are not intended to limit the scope of the present disclosure.

### BRIEF DESCRIPTION OF THE DRAWINGS

FIG. 1 depicts the PROMIS fatigue T score change from baseline for both the combination and SOC control at Week 14.

FIG. 2 depicts the NRS fatigue score change from baseline for both the combination and SOC control at Week 14.

FIG. 3 depicts the NRS pain scale change from baseline for both the combination and SOC control at Week 14.

FIG. 4 shows the PGIC responder rate at Week 14.

FIG. 5 depicts the OISA, OISAS change from baseline for both the combination and SOC control at Week 14.

FIG. 6 depicts the OIDAS change from baseline for both the combination and SOC control at Week 14.

FIG. 7 depicts the occurrence of symptoms of subjects enrolled in the open-label trial of Example 3.

FIG. 8 compares the average percent improvement in fatigue following treatment with a combination of nirmatrelvir and ritonavir (PAXLOVID™) plus a combination of 1.5 g valacyclovir and 200 mg celecoxib (IMC-2) or the combination of valacyclovir and celecoxib alone.

FIG. 9 compares the average percent improvement in brain fog following treatment with the combination of nirmatrelvir and ritonavir (PAXLOVID™) plus the combination of valacyclovir and celecoxib (IMC-2) or the valayclovir/celecoxib combination alone.

FIG. 10 compares the average percent improvement in dysautonomia following treatment with the combination of nirmatrelvir and ritonavir (PAXLOVID™) plus the combination of valacyclovir and celecoxib (IMC-2) or the valayclovir/celecoxib combination alone.

FIG. 11 compares the average percent improvement in various symptoms of long-COVID following treatment with the combination of nirmatrelvir and ritonavir (PAXLOVID™) plus the combination of valacyclovir and celecoxib (IMC-2) or the valayclovir/celecoxib combination alone.

FIG. 12 depicts the occurrence of symptoms of subjects enrolled in the open-label trial of Example 4.

FIG. 13 compares the average percent improvement in fatigue following treatment with a combination of nirmatrelvir and ritonavir (PAXLOVID™) plus a combination of valacyclovir and celecoxib (IMC-2) or the combination of valacyclovir and celecoxib alone.

FIG. 14 compares the average percent improvement in brain fog following treatment with the combination of nirmatrelvir and ritonavir (PAXLOVID™) plus the combination of valacyclovir and celecoxib (IMC-2) or the valayclovir/celecoxib combination alone.

FIG. 15 compares the average percent improvement in dysautonomia following treatment with the combination of nirmatrelvir and ritonavir (PAXLOVID™) plus the combination of valacyclovir and celecoxib (IMC-2) or the valayclovir/celecoxib combination alone.

FIG. 16 compares the average percent improvement in various symptoms of long-COVID following treatment with the combination of nirmatrelvir and ritonavir (PAXLOVID™) plus the combination of valacyclovir and celecoxib (IMC-2) or the valayclovir/celecoxib combination alone.

FIG. 17 compares the primary endpoint and secondary endpoints at 120-day versus the 305-day long-term data.

### DETAILED DESCRIPTION

The following description is merely exemplary in nature and is not intended to limit the present disclosure, application, or uses.

### A. Definitions

The term “pharmaceutically acceptable” means suitable for use in pharmaceutical preparations, generally considered as safe for such use, officially approved by a regulatory agency of a national or state government for such use, or being listed in the U. S. Pharmacopoeia or other generally recognized pharmacopoeia for use in animals, and more particularly in humans.

The term “therapeutically-effective amount” refers to an amount of a compound that, when administered to a subject for treating a disease, is sufficient to effect treatment for the disease. “Therapeutically effective amount” can vary depending on the compound, the disease and its severity, the age, the weight, etc. of the subject to be treated.

The term “COX-2 inhibitor” refers to a cyclooxygenase-2 inhibitor, which is any pharmaceutically acceptable compound that inhibits the enzyme cyclooxygenase-2.

The term “COX-1 inhibitor” refers to a cyclooxygenase-1 inhibitor, which is any pharmaceutically acceptable compound that inhibits the enzyme cyclooxygenase-1.

The term “PAXLOVID™” refers to the combination of nirmatrelvir with ritonavir.

The term “HSV-1” refers to herpes simplex virus-1.

The terms “prevent,” “prevention,” or “preventing” refer to either preventing the onset of preclinically evident condition altogether or preventing the onset of a preclinical evident stage of the condition in a subject. Prevention includes, but is not limited to, prophylactic treatment of a subject at risk of developing a condition.

The term “treat” (and corresponding terms “treatment” and “treating”) includes palliative, restorative, and preventative treatment of a subject. The term “palliative treatment” refers to treatment that eases or reduces the effect or intensity of a condition in a subject without curing the condition. The term “preventative treatment” (and the corresponding term “prophylactic treatment”) refers to treatment that prevents the occurrence of a condition in a subject. The term “restorative treatment” refers to treatment that halts the progression of, reduces the pathologic manifestations of, or entirely eliminates a condition in a subject.

The term “synergy” and its related terms (e.g., synergistically effective) refers to a combination of active ingredients (e.g., a combination of one or more of an antiviral compound, a COX-2 inhibitor, nirmatrelvir, and ritonavir) which induces an effect that is greater than additive or the sum of each component part.

COVID-19 refers to the severe acute respiratory syndrome caused by infection with the RNA virus SARS-COV-2. Long COVID, also known as “long haul COVID,” refers to the condition wherein subjects experience the effects of the infection for an extended period of time (e.g., months or years) following recovery from the initial infection with SARS-COV-2. Such long-term effects include, but are not limited to, severe chronic fatigue that is not due to ongoing exertion or a medical condition and that significantly interferes with daily activities.

The term “CFS” refers to chronic fatigue syndrome. In some embodiments, the CFS is myalgic encephalomyelitis/CFS (ME/CFS)

The term “cognitive dysfunction”, also referred to as “brain fog”, “mental fog”, or “impaired cognition”, refers to the loss or impairment of intellectual function (such as thinking, remembering, or reasoning) of sufficient severity to interfere with daily functioning.

The terms “famcyclovir” and “famciclovir” refer to the same antiviral compound.

The terms “valacyclovir” and “valaciclovir” refer to the same antiviral compound.

The terms “acyclovir” and “aciclovir” refer to the same antiviral compound.

The term “QD” refers to once a day.

The term “BID” refers to two times a day.

The term “TID” refers to three times a day.

The term “QID” refers to four times a day.

The term PO refers to oral administration.

The term “Likert Survey” (and the corresponding term “Likert Scale”) refers to a questionnaire which asks subjects the extent to which they agree or disagree with a statement, using a five-point scale.

The term “combination therapy” (or “co-therapy”), in defining use of an antiviral compound, a COX-2 inhibitor, and one or more additional agents such as nirmatrelvir and ritonavir, as described herein, is intended to embrace administration of each agent in a sequential manner in a regimen that will provide beneficial effects of the drug combination, and is intended as well to embrace co-administration of these agents in a substantially simultaneous manner, such as by oral ingestion of a single capsule having a fixed ratio of these active agents or ingestion of multiple, separate capsules for each agent. “Combination therapy” will also include simultaneous or sequential administration by intravenous, intramuscular, or other parenteral routes into the body, including direct absorption through mucous membrane tissues, as found in the sinus passages. Sequential administration also includes drug combination where the individual elements may be administered at different times and/or by different routes but which act in combination to provide a beneficial effect. It is expected that this combination therapy of an antiviral compound, a COX-2 inhibitor, and one or more additional agents will result in co-action of the active ingredients, providing a pharmacokinetic interaction, or a pharmacodynamic interaction, or both, where the compounds are administered either simultaneously or sequentially, to permit such co-action.

### B. Clinical Observations

The present invention is to be understood as embracing treatment of COVID-19, including long COVID. U.S. Pat. No. 8,809,351 reports that a combination of a COX-2 inhibitor and an antiviral agent (e.g., famciclovir and celecoxib or valacyclovir and celecoxib) are useful for treating functional somatic syndromes (FSS), including but not limited to fibromyalgia, as well as pain and associated functional symptoms associated with fibromyalgia. Patients with fibromyalgia were observed to display a variety of symptoms, including but not limited to fatigue, insomnia, depression, allodynia, headaches, irritable bowel syndrome, sensitivity to light, numbness, and anxiety. Stress often exacerbates the symptoms. While the etiology and pathogenesis of FSS is not clearly understood, a combination of interactions among external stressors, neurotransmitters, hormones, the immune system, and the sympathetic nervous system, appear to be involved. In particular, Herpes virus is hypothesized to play a major role in fibromyalgia and related functional somatic syndromes.

Herpes viruses including HSV-1 and HSV-2 are unique because after the initial infection they may remain dormant in tissue until conditions are sufficient for a reactivation. Physiologic stressors associated with this reactivation process may initiate the synthesis and release of host peptides and hormones of the sympathetic nervous system and the hypothalamic-pituitary-adrenal axis. Most viruses in this class infrequently reactivate. Both HSV-1 and HSV-2 reactivate often enough to create the milieu needed for development of a chronic debilitating illness. HSV-2 cycles only once or twice a year and is an unlikely candidate for chronic illness. HSV-1, however, cycles frequently enough, on average 4 times a year, and as often as monthly, to result in a slowly developing debilitating illnesses. Without being bound by theory, U.S. Pat. No. 8,809,351 theorizes that after many reactivations, a neuronal cell body dies due to apoptosis and that the ganglion undergoes destruction in the regions governing the first inoculation site.

The existence of Herpes virus in one or more ganglia may directly or indirectly affect the central nervous system (CNS), hypothalamic pituitary axes (HPA) and immune system. Dysregulation of pain processing within the CNS may lead to an amplified perception of pain and other sensory stimuli. This phenomenon, often referred to as central sensitization or augmentation, results from changes in the properties of neurons in the CNS where pain is no longer coupled, as acute nociceptive pain is, to the presence, intensity, or duration of noxious peripheral stimuli. Neurotransmitters such as glutamate, Substance P, serotonin, norepinephrine, dopamine, brain-derived neurotrophic factor (BDNF), and gamma aminobutyric acid (GABA) are activated in chronic pain and depression. Substance P, cerebrospinal fluid levels and serum concentration of BDNF have been consistently higher in patients with fibromyalgia compared with controls. Patients with fibromyalgia also have an abnormal dopamine response to pain. Recent data suggest a putative role of pro-inflammatory cytokines, including interleukin-1-beta, tumor necrosis factor-alpha (TNFα), IL-6 and IL-8, in the pathogenesis of fibromyalgia and the modulation of symptoms [M. DiFranco, et al., Ann. N. Y. Acad. Sci. 1193 (1), 84-90 (2010)]. Hence pain, as the defining characteristic of fibromyalgia, is not due to tissue damage or inflammation and is thus fundamentally different from rheumatic disorders and many other pain conditions as these conditions cause inflammation in the joints and tissues. Herpes virus has developed various immune evasion mechanisms which prove to be a particular challenge for the immune system. Cytokines and cytokine-induced genes are important for the ability of any organism to raise an antiviral response. Understanding the immune mediators and their possible role in fibromyalgia may be the most daunting obstacles in understanding the disorder. Gur and Oktayoglu [A. Gur and P. Oktayoglu, Curr. Pain Headache Rep. 12(3), 175-181(2008)] explain how cytokines related to acute or repetitive tissue injuries may be responsible for long-term activation of spinal cord glia and dorsal horn neurons, thus resulting in central sensitization. The immune system responds to stressors by causing certain immune cells to secrete the pro-inflammatory cytokines IL-1 and IL-6. Both cytokines are involved in inflammation, and IL-6 is thought to worsen the symptoms of autoimmune disease and fibromyalgia [L. Vanderhaeghe, Total Health 23, 34-35(2001)]. IL-1 and IL-6 are both correlated with the development and severity of Alzheimer's disease. [Su, et al., Neurosci. Bull., 32(5), 469-480 (2016)]

Various studies confirm that isoforms of COX-1 and COX-2 are critical for efficient viral replication. In one study Ray and Enquist showed that simultaneous inhibition of COX-1 and COX-2 caused a dramatic reduction of viral yield after HSV-1 infection [N. Ray and L. Enquist, J. Virol. 78, 3489-3501 (2004)]. Hill, et al., used microarrays to analyze gene expression in the trigeminal ganglion of mice infected with latent HSV-1, and found COX-2 gene expression significantly up regulated after reactivation [J. Hill, et al., Virus Genes 23, 273-280 (2001)]. Gebhardt reported that the selective COX-2 inhibitor celecoxib can suppress hyperthermic stress-induced herpes viral reactivation in the nervous system of mice [B. Gebhardt, et al., J. Ocul. Pharmacol. Ther. 21, 114-120 (2005)].

Functional somatic syndromes (FSS) may be defined as conditions “characterized by patterns of persistent bodily complaints for which adequate examination does not reveal sufficiently explanatory structural or other specified pathology” [P. Henningsen, et al. (2007) Lancet 369, 946-954]. A diverse number of conditions are commonly described as FSS, including: fibromyalgia, irritable bowel syndrome, chronic fatigue syndrome, premenstrual syndrome, non-ulcer dyspepsia, chronic pain, chronic pelvic pain, hypoglycemia, low back pain, sick building syndrome, Gulf War syndrome, tension headache, tempo-mandibular joint disorder, repetitive strain injury, multiple chemical sensitivity, interstitial cystitis, chronic Lyme disease, depression, post-traumatic stress disorder (PTSD), chronic anxiety disorder, food hypersensitivity, and brain fog or cognitive dysfunction.

Despite the broad range of FSS conditions, these disease states may have a common etiology rather than being distinct syndromes. Wessely and colleagues concluded on the basis of a literature review that there was substantial overlap between these conditions and that their similarities were greater than their differences, proposing the concept of a general functional somatic syndrome [S. Wessely, et al. (1999) Lancet 354, 936-939]. A common etiology for FSS was also explored by Bland, who pointed out that when the allostatic load, the combined external and internal stress, exceeds the ability of the patient to maintain allostasis, alterations in function occur giving rise to symptomatic FSS [J. Bland (2008) Alt. Therapies 14, 14-16.] Somatic syndrome disorder further classifies IBS, fibromyalgia, and ME/CFS and other conditions.

U.S. Pat. No. 8,809,351 provides additional support for the concept that Herpes virus is a common etiological stressor that gives rise to fibromyalgia and FSS. Briefly, U.S. Pat. No. 8,809,351 disclosed that of nineteen patients suffering from fibromyalgia, eighteen showed the presence of HSV-1 DNA. HSV-1 infection was confirmed through the detection of the virus protein ICP8 in biopsies of the positive patients. U.S. Pat. No. 8,809,351 further demonstrated that administration of the combination of famciclovir and celecoxib effectively treated fibromyalgia, brain fog, inflammatory bowel disease (IBS), chronic fatigue syndrome (CFS), chronic pain, and brain fog.

Herpes virus markedly decreases both natural killer (NK) and CD8+ T cell activity, both of which are critical components of the immune response to viral infections. In this way, Herpes virus may be an immunosuppressing infection. Downregulation of NK and CD8+ T cell activity has been shown in chronic pain, ME/CFS, and long COVID. Without being bound by theory, it is hypothesized that following an acute SARS-COV2 infection, a subsequent Herpes virus infection (or a coinfection of Herpes virus and SARS-COV2) persists as a nociceptive low-grade illness that results in long COVID. Given the similarity of symptoms of long COVID with ME/CFS, it is theorized that administration of an antiviral agent and a COX-2 inhibitor can be an effective treatment for long COVID.

### C. Pharmaceutical Compositions

The compounds of the present invention can be administered in a unit dosage from. If desired, multiple doses per day of the unit dosage form can be used to increase the total daily dose. Combinations of a COX-2 inhibitor and an antiviral compound are disclosed in U.S. Pat. No. 8,809,351, which is incorporated herein by reference. The present disclosure provides a combination of a COX-2 inhibitor, an antiviral compound, and one or more additional active ingredients, such as a combination of nirmatrelvir and ritonavir, molnupiravir, BCG vaccine, and/or ivermectin. In various embodiments, the combination of the present disclosure is one selected from the specific combinations disclosed in U.S. Pat. No. 8,809,351 combined with one or more additional active ingredients, such as a combination of nirmatrelvir and ritonavir, molnupiravir, BCG vaccine, or ivermectin. Particular embodiments include a combination of celecoxib, valacyclovir, nirmatrelvir, and ritonavir and a combination of celecoxib, famciclovir, nirmatrelvir, and ritonavir.

In another embodiment, there is provided a pharmaceutical composition, as described herein, wherein the amount of antiviral compound is present in a unit dosage form from about 250 mg to about 2000 mg. The antiviral compound may be administered once a day or twice daily.

In another embodiment, there is provided a pharmaceutical composition, as described herein, wherein the amount of antiviral compound is present in a unit dosage form from about 250 mg to about 1000 mg.

In another embodiment, there is provided a pharmaceutical composition, as described herein, wherein the amount of antiviral compound is present in a unit dosage form from about 250 mg to about 500 mg.

In another embodiment, there is provided a pharmaceutical composition, as described herein, wherein the antiviral compound is a guanine analog antiviral compound.

In another embodiment, there is provided a pharmaceutical composition, as described herein, wherein the antiviral compound is selected from the group consisting of famciclovir, valacyclovir, and acyclovir.

In another embodiment, there is provided a pharmaceutical composition, as described herein, wherein the antiviral compound is famciclovir.

In another embodiment, there is provided a pharmaceutical composition, as described herein, wherein the amount of famciclovir is present in a unit dosage form from about 250 mg to about 1000 mg.

In another embodiment, there is provided a pharmaceutical composition, as described herein, wherein the antiviral compound is valacyclovir.

In another embodiment, there is provided a pharmaceutical composition, as described herein, wherein the amount of valacyclovir is present in a unit dosage form from about 1000 mg to about 2000 mg.

In another embodiment, there is provided a pharmaceutical composition, as described herein, wherein the antiviral compound is acyclovir.

In another embodiment, there is provided a pharmaceutical composition, as described herein, wherein the amount of acyclovir is present in a unit dosage form from about 400 mg to about 1600 mg.

In another embodiment, there is provided a pharmaceutical composition, as described herein, wherein the amount of COX-2 inhibitor is present in a unit dosage form from about 7.5 mg to about 600 mg. The COX-2 inhibitor may be administered once a day or twice daily.

In another embodiment, there is provided a pharmaceutical composition, as described herein, wherein the amount of COX-2 inhibitor is present in a unit dosage form from about 15 mg to about 300 mg.

In another embodiment, there is provided a pharmaceutical composition, as described herein, wherein the amount of COX-2 inhibitor is present in a unit dosage form from about 50 mg to about 200 mg.

In another embodiment, there is provided a pharmaceutical composition, as described herein, wherein the COX-2 inhibitor is selected from the group consisting of celecoxib, meloxicam, and a diclofenac-misoprostol combination.

In another embodiment, there is provided a pharmaceutical composition, as described herein, wherein the COX-2 inhibitor is celecoxib.

In another embodiment, there is provided a pharmaceutical composition, as described herein, wherein the amount of celecoxib is present in a unit dosage form from about 50 mg to about 600 mg.

In another embodiment, there is provided a pharmaceutical composition, as described herein, wherein the COX-2 inhibitor is meloxicam.

In another embodiment, there is provided a pharmaceutical composition, as described herein, wherein the amount of meloxicam is present in a unit dosage form from about 7.5 mg to about 15 mg.

In another embodiment, there is provided a pharmaceutical composition, as described herein, wherein the COX-2 inhibitor is a diclofenac-misoprostol combination.

In another embodiment, there is provided a pharmaceutical composition, as described herein, wherein the amount of diclofenac is present in a unit dosage form from about 50 mg to about 200 mg and the amount of misoprostol is present in a unit dosage form from about 200 μg to about 800 μg.

In another embodiment, there is provided a pharmaceutical composition, as described herein, wherein the one or more additional active ingredients is selected from the group consisting of a combination of nirmatrelvir and ritonavir, molnupiravir, BCG vaccine, and ivermectin.

In another embodiment, there is provided a pharmaceutical composition, as described herein, wherein the one or more additional active ingredients is a combination of nirmatrelvir and ritonavir, wherein the nirmatrelvir is present in a unit dosage form from about 50 mg to about 450 mg and the ritonavir is present in a unit dosage form from about 50 mg to about 200 mg. The nirmatrelvir and ritonavir may be administered to achieve a daily dosage range of about 100 mg to about 900 mg and about 100 mg to about 400 mg, respectively, wherein the nirmatrelvir and ritonavir may be administered once a day or twice daily.

In another embodiment, there is provided a pharmaceutical composition, as described herein, wherein the one or more additional active ingredients is molnupiravir, which may be present in a unit dosage form from about 200 mg to about 1600 mg. The molnupiravir may be administered to achieve a daily dosage range of about 400 mg to about 3200 mg, which may be administered once a day or twice daily.

In another embodiment, there is provided a pharmaceutical composition, as described herein, wherein the one or more additional active ingredients is BCG vaccine or ivermectin.

In one embodiment, there is provided a combination, comprising a therapeutically-effective amount of valacyclovir, a therapeutically-effective amount of celecoxib, a therapeutically effective amount of nirmatrelvir, and a therapeutically effective amount of ritonavir, wherein the amount of valacyclovir is present in a unit dosage form from about 750 mg to about 2000 mg, wherein the amount of celecoxib is present in a unit dosage form from about 200 mg to about 800 mg, wherein the amount of nirmatrelvir is present in a unit dosage form from about 50 mg to about 450 mg, and wherein the amount of ritonavir is present in a unit dosage form from about 50 mg to about 200 mg. In a further embodiment, the valacyclovir, celecoxib, nirmatrelvir, and ritonavir may be administered once a day or twice daily.

In another embodiment, there is provided a combination, as described herein, wherein the amount of valacyclovir is present in a unit dosage form from about 750 mg to about 1050 mg.

In another embodiment, there is provided a combination, as described herein, wherein the amount of valacyclovir is present in a unit dosage form from about 1050 mg to about 1500 mg.

In another embodiment, there is provided a combination, as described herein, wherein the amount of valacyclovir is present in a unit dosage form selected from the group consisting of about 750 mg, about 1050 mg, about 1250 mg, about 1500 mg, and about 2000 mg.

In another embodiment, there is provided a combination, as described herein, wherein the amount of valacyclovir is present in a unit dosage form of about 750 mg or about 1250 mg.

In another embodiment, there is provided a combination, as described herein, wherein the amount of celecoxib is present in a unit dosage form from about 100 mg to about 400 mg.

In another embodiment, there is provided a combination, as described herein, wherein the amount of celecoxib is present in a unit dosage form from about 400 mg to about 800 mg.

In another embodiment, there is provided a combination, as described herein, wherein the amount of celecoxib is present in a unit dosage form selected from the group consisting of about 100 mg, about 200 mg, about 400 mg, and about 800 mg.

In another embodiment, there is provided a combination, as described herein, wherein the amount of celecoxib is present in a unit dosage form of about 200 mg or about 400 mg.

In another embodiment, there is provided a combination, as described herein, wherein the amount of valacyclovir is present in a unit dosage form of about 750 mg or about 1250 mg, and wherein the amount of celecoxib is present in a unit dosage form of about 200 mg or about 400 mg.

In another embodiment, the antiviral compound, the COX-2 inhibitor, the nirmatrelvir and the ritonavir are present in amounts that are synergistically effective for treating long-COVID, post-acute infection syndrome (PAIS), and/or orthostatic intolerance. In a particular embodiment, the synergistically effective composition comprises about 750 to about 2000 mg valacyclovir, about 200 to about 800 mg celecoxib, about 50 mg to about 450 mg nirmatrelvir, and about 50 mg to about 200 mg ritonavir. In another particular embodiment, the synergistically effective composition comprises about 750 to about 1500 mg famciclovir, about 200 to about 800 mg celecoxib, about 50 mg to about 450 mg nirmatrelvir, and about 50 mg to about 200 mg ritonavir. In a further embodiment, the valacyclovir for famciclovir, celecoxib, nirmatrelvir, and ritonavir may be administered once a day or twice daily.

In one embodiment, there is provided a kit presentation, comprising a therapeutically-effective amount of valacyclovir or famciclovir in a first unit dosage form, a therapeutically-effective amount of celecoxib in a second unit dosage form, and one or more additional active ingredients in additional unit dosage forms, wherein the first, second, and additional unit dosage forms are separately enclosed in one or more containers, arranged in a single package or dispensing device, optionally comprising directions on how to use kit components suitable for administration to obtain a therapeutic outcome. The additional dosage forms may be a third unit dosage form comprising nirmatrelvir and a fourth unit dosage from comprising ritonavir. Alternatively, the additional unit dosage from may be a third unit dosage from comprising molnupiravir, BCG vaccine, or ivermectin.

In another embodiment, there is provided a kit presentation, as described herein, wherein the amount of valacyclovir or famciclovir is present in a unit dosage form from about 750 mg to about 2000 mg, wherein the amount of celecoxib is present in a unit dosage form from about 100 mg to about 800 mg, wherein the amount of nirmatrelvir is present in a unit dosage form from about 50 mg to about 450 mg, and wherein the amount of ritonavir is present in a unit dosage form from about 50 mg to about 200 mg. In an embodiment, each of the components of the kit is present in a synergistically effective amount.

In another embodiment, there is provided a dosage form wherein the total amount of drug present may be from about 0.05% to about 95% by weight, more typically from about 2% to about 50% by weight of the dosage form.

For the treatment of the conditions referred to herein, the compounds described herein can be administered as described below.

### Oral Administration

The compounds of the present disclosure may be administered orally, including by swallowing, so that the compound enters the gastrointestinal tract, or absorbed into the blood stream directly from the mouth (e.g., buccal or sublingual administration).

Suitable compositions for oral administration include solid formulations such as tablets, lozenges and capsules, which can contain liquids, gels, or powders.

Compositions for oral administration may be formulated as immediate or modified release, including delayed or sustained release, optionally with enteric coating.

Liquid formulations can include solutions, syrups, and suspensions, which can be used in soft or hard capsules. Such formulations may include a pharmaceutically acceptable carrier, for example, water, ethanol, polyethylene glycol, cellulose, or an oil. The formulation may also include one or more emulsifying agents and/or suspending agents.

Tablets may contain a disintegrant, comprising from about 0.5% to about 35% by weight, more typically from about 2% to about 25% of the dosage form. Examples of disintegrants include methyl cellulose, sodium or calcium carboxymethyl cellulose, croscarmellose sodium, polyvinylpyrrolidone, hydroxypropyl cellulose, starch and the like.

Suitable lubricants, for use in a tablet, may be present in amounts from about 0.1% to about 5% by weight, and include calcium, zinc or magnesium stearate, sodium stearyl fumarate and the like.

Suitable binders, for use in a tablet, include gelatin, polyethylene glycol, sugars, gums, starch, hydroxypropyl cellulose and the like. Suitable diluents, for use in a tablet, include mannitol, xylitol, lactose, dextrose, sucrose, sorbitol and starch.

Suitable surface active agents and glidants, for use in a tablet, may be present in amounts from about 0.1% to about 3% by weight, and include polysorbate 80, sodium dodecyl sulfate, talc and silicon dioxide.

### Parenteral Administration

Compounds of the present disclosure may be administered directly into the blood stream, muscle, or internal organs. Suitable means for parenteral administration include intravenous, intra-muscular, subcutaneous intraarterial, intraperitoneal, intrathecal, intracranial, and the like. Suitable devices for parenteral administration include injectors (including needle and needle-free injectors) and infusion methods.

Compositions for parenteral administration may be formulated as immediate or modified release, including delayed or sustained release.

Most parenteral formulations are aqueous solutions containing excipients, including salts, buffering agents and carbohydrates.

Parenteral formulations may also be prepared in a dehydrated form (e.g., by lyophilization) or as sterile non-aqueous solutions. These formulations can be used with a suitable vehicle, such as sterile water. Solubility-enhancing agents may also be used in preparation of parenteral solutions.

### Topical Administration

Compounds of the present disclosure may be administered topically to the skin or transdermally. Formulations for this topical administration can include lotions, solutions, creams, gels, hydrogels, ointments, foams, implants, patches, and the like. Pharmaceutically acceptable carriers for topical administration formulations can include water, alcohol, mineral oil, glycerin, polyethylene glycol, and the like. Topical administration can also be performed by electroporation, iontophoresis, phonophoresis, and the like.

Compositions for topical administration may be formulated as immediate or modified release, including delayed or sustained release.

### Kits

Compound combinations of the present disclosure, wherein component A is an antiviral compound, component B is a COX-2 inhibitor, and additional components are the one or more additional active ingredients. When the one or more additional active ingredients is the combination of nirmatrelvir and ritonavir, component C is nirmatrelvir and component D is ritonavir. When the one or more additional active ingredients is molunpiravir, BCG vaccine, or ivermectin, each may be independently present as component C. As described herein, the components can be provided in a kit presentation, comprising an arrangement of components A, B, C, and D in association with each other, as in a single package or in a drug dispensing device. Such kit presentations, used by a patient, may be dispensed by a hospital-formulary, retail pharmacist, or prescribing-physician.

In one example, the kit may comprise a single package, with therapeutically effective doses of components A, B, C, and D, in the form of tablets or capsules in separate containers (e.g., bottles) held separately, as in a tray, and bound together in a single package using, for example, shrink wrap, tape, or a plastic or cardboard box enclosing the components.

In another example, separate, therapeutically-effective doses of combination components A, B, C, and D in the form of tablets or capsules, may be presented as co-packaged, in a single blister pack.

In another example, the kit presentation may provide therapeutically-effective doses of combination components A, B, C, and D in the form of tablets or capsules, which are co-dispensed from a device which delivers the components from a storage receptacle using, for example, one or more levers to co-dispense individual dose forms of components A, B, and C to be administered in combination.

The kit presentation may also be used for parenteral administration of dose forms of Components A, B, C, and D. For example, individual doses of components A, B, C, and D in the form of lyophilized powders, either separately or with components A, B, C, and D mixed together in therapeutically-effective doses, arranged in a package also comprising separately contained vials of sterile water or buffer solution, and optionally a sterile packaged syringe for administration of the dose combination following dissolution.

The kit presentation may further comprise directions, in compliance with approved instructions from a government agency (e.g., U.S. FDA), on how to use the kit components suitable for administration to obtain a therapeutic outcome.

### D. Methods of Treatment

The present disclosure further provides methods for treating a condition in a subject having or susceptible to having such a condition, by administering to the subject a therapeutically-effective amount of the compounds as described above. In one embodiment, the treatment is preventative treatment. In another embodiment, the treatment is palliative treatment. In another embodiment, the treatment is restorative treatment.

In one embodiment there is provided a method to treat a subject susceptible to or afflicted with long COVID, the method comprising administering to the subject a therapeutically-effective amount of a COX-2 inhibitor, a therapeutically-effective amount of an antiviral compound, a therapeutically-effective amount of one or more additional active ingredients, such as a combination of a therapeutically effective amount of nirmatrelvir and a therapeutically-effective amount of ritonavir, a therapeutically effective amount of molnupiravir, a therapeutically effective amount of BCG vaccine, or a therapeutically effective amount of ivermectin. The antiviral component may be administered in a total daily dose range from about 250 mg to about 2000 mg. The COX-2 inhibitor component may be administered in a total daily dose range from about 15 mg to about 800 mg. The nirmatrelvir component may be administered in a total daily dose range from about 100 mg to about 900 mg, delivered in a single dose or in two daily doses. The ritonavir component may be administered in a total daily dose range from about 100 mg to about 400 mg, delivered in a single dose or in two daily doses. In a specific embodiment, the COX-2 inhibitor is celecoxib, the antiviral compound is famciclovir, and the one or more additional active ingredients is a combination of nirmatrelvir and ritonavir. In another specific embodiment, the COX-2 inhibitor is celecoxib, the antiviral compound is valacyclovir, and the one or more additional active ingredients is a combination of nirmatrelvir and ritonavir.

In another embodiment, there is provided a method to treat a subject susceptible to or afflicted with a post-COVID19 illness including a post-acute infection syndrome (PAIS). In various embodiments, the method is used to treat a PAIS which is a sequelae of infection selected from the group consisting of SARS COV-2 infection, Epstein-Barr virus infection, Ross River virus infection, human herpesvirus-6 infection, Varicella-zoster virus infection, Ebola virus infection, West Nile virus infection, Dengue virus infection, parvovirus infection, Borrelia burgdorferi infection, Coxiella burnetiid infection, and Mycoplasma pneumoniae infection. The method may comprise administering to the subject a therapeutically-effective amount of a COX-2 inhibitor, a therapeutically-effective amount of an antiviral compound, and a therapeutically-effective amount of a one or more additional active ingredients, such as a combination of a therapeutically-effective amount of nirmatrelvir and a therapeutically-effective amount of ritonavir, a therapeutically-effective amount of molnupiravir, a therapeutically-effective amount of BCG vaccine, or a therapeutically effective amount of ivermectin. The antiviral component may be administered in a total daily dose range from about 250 mg to about 2000 mg. The COX-2 inhibitor component may be administered in a total daily dose range from about 15 mg to about 800 mg. The nirmatrelvir component may be administered in a total daily dose range from about 100 mg to about 900 mg, delivered in a single dose or in two doses. The ritonavir component may be administered in a total daily dose range from about 100 mg to about 400 mg, delivered in a single dose or in two doses. In a specific embodiment, the COX-2 inhibitor is celecoxib, the antiviral compound is famciclovir, and the one or more additional active ingredients is a combination of nirmatrelvir and ritonavir. In another specific embodiment, the COX-2 inhibitor is celecoxib and the antiviral compound is valacyclovir, and the one or more additional active ingredients is a combination of nirmatrelvir and ritonavir.

In another embodiment, there is provided a method to treat symptoms of orthostatic intolerance in a subject suffering from PAIS. In various embodiments, the method is used to treat a PAIS which is a sequelae of infection selected from the group consisting of SARS COV-2 infection, Epstein-Barr virus infection, Ross River virus infection, human herpesvirus-6 infection, Varicella-zoster virus infection, Ebola virus infection, West Nile virus infection, Dengue virus infection, parvovirus infection, Borrelia burgdorferi infection, Coxiella burnetiid infection, and Mycoplasma pneumoniae infection. The symptoms may be dizziness, feeling faint, light-headedness, feeling about to blackout, blurred vision, tunnel vision, seeing spots, weakness, fatigue, trouble concentrating, head and neck discomfort, difficulty standing for a short time, difficulty standing for a long time, difficulty walking for a short time, and/or difficulty walking for a long time. The method may comprise administering to the subject a therapeutically-effective amount of a COX-2 inhibitor, in a dose weight ratio range from about one-to-one to about five hundred-to-one of the antiviral compound to the COX-2 inhibitor. In a specific embodiment, the COX-2 inhibitor is celecoxib and the antiviral compound is famciclovir. In another specific embodiment, the COX-2 inhibitor is celecoxib and the antiviral compound is valacyclovir.

In one embodiment there is provided a method to treat a subject susceptible to or afflicted with orthostatic intolerance, the method comprising administering to the subject a therapeutically-effective amount of a COX-2 inhibitor, a therapeutically-effective amount of an antiviral compound, and a therapeutically-effective amount of one or more additional active ingredients, such as a combination of a therapeutically-effective amount of nirmatrelvir and a therapeutically-effective amount of ritonavir, a therapeutically-effective amount of molnupiravir, a therapeutically-effective amount of BCG vaccine, or a therapeutically-effective amount of ivermectin. The antiviral component may be administered in a total daily dose range from about 250 mg to about 2000 mg. The COX-2 inhibitor component may be administered in a total daily dose range from about 15 mg to about 800 mg. The nirmatrelvir component may be administered in a total daily dose range from about 100 mg to about 900 mg, delivered once a day or twice daily. The ritonavir component may be administered in a total daily dose range from about 100 mg to about 400 mg, delivered once a day or twice daily. In a specific embodiment, the COX-2 inhibitor is celecoxib, the antiviral compound is famciclovir, and the one or more additional active ingredients is a combination of nirmatrelvir and ritonavir. In another specific embodiment, the COX-2 inhibitor is celecoxib, the antiviral compound is valacyclovir, and one or more additional active ingredients is a combination of nirmatrelvir and ritonavir.

In another embodiment, there is provided a method, as described herein, wherein the dose weight ratio range is from about one-to-one to about one hundred-to-one of the antiviral compound to the COX-2 inhibitor.

In another embodiment, there is provided a method, as described herein, wherein the amount of antiviral compound is present in a unit dosage form from about 250 mg to about 2000 mg.

In another embodiment, there is provided a method, as described herein, wherein the amount of antiviral compound is present in a unit dosage form from about 250 mg to about 1000 mg.

In another embodiment there is provided a method, as described herein, wherein the amount of antiviral compound is present in a unit dosage form from about 250 mg to about 500 mg.

In another embodiment, there is provided a method, as described herein, wherein the amount of famciclovir is present in a unit dosage form from about 250 mg to about 1000 mg.

In another embodiment, there is provided a method, as described herein, wherein the amount of valacyclovir is present in a unit dosage form from about 1000 mg to about 2000 mg.

In an alternative embodiment, there is provided a method, as described herein, wherein the antiviral compound is acyclovir.

In another alternative embodiment, there is provided a method, as described herein, wherein the amount of acyclovir is present in a unit dosage form from about 400 mg to about 1600 mg.

In another embodiment, there is provided a method, as described herein, wherein the amount of COX-2 inhibitor is present in a unit dosage form from about 7.5 mg to about 600 mg.

In another embodiment, there is provided a method, as described herein, wherein the amount of COX-2 inhibitor is present in a unit dosage form from about 15 mg to about 300 mg.

In another embodiment, there is provided a method, as described herein, wherein the amount of COX-2 inhibitor is present in a unit dosage form from about 50 mg to about 200 mg.

In an alternative embodiment, there is provided a method, as described herein, wherein the COX-2 inhibitor is meloxicam or a diclofenac-misoprostol combination.

In another embodiment, there is provided a method, as described herein, wherein the amount of celecoxib is present in a unit dosage form from about 50 mg to about 600 mg.

In another embodiment, there is provided a method, as described herein, wherein the COX-2 inhibitor is meloxicam.

In another embodiment, there is provided a method, as described herein, wherein the amount of meloxicam is present in a unit dosage form from about 7.5 mg to about 15 mg.

In another embodiment, there is provided a method, as described herein, wherein the COX-2 inhibitor is a diclofenac-misoprostol combination.

In another embodiment, there is provided a method, as described herein, wherein the amount of diclofenac is present in a unit dosage form from about 50 mg to about 200 mg and the amount of misoprostol is present in a unit dosage form from about 200 μg to about 800 μg.

In another embodiment, there is provided a method, as described herein, wherein the amount of nirmatrelvir is present in a unit dosage form from about 50 mg to about 450 mg.

In another embodiment, there is provided a method, as described herein, wherein the amount of ritonavir is present in a unit dosage form from about 50 mg to about 200 mg.

In one embodiment, there is provided a method to treat a subject susceptible to or afflicted with long COVID, the method comprising administering to the subject a therapeutically-effective combination of famciclovir and celecoxib, wherein the amount of famciclovir is administered in a total daily dose range from about 250 mg to about 1000 mg, and wherein the amount of celecoxib is administered in a total daily dose range from about 200 mg to about 800 mg, wherein the amount of nirmatrelvir is administered in a total daily dose range from about 100 mg to about 900 mg, which may be delivered once a day or twice daily, and wherein the amount of ritonavir is administered in a total daily dose range from about 100 mg to about 400 mg, which may be delivered once a day or twice daily, and wherein the combination administered produces no substantial adverse event.

In one embodiment, there is provided a method to treat a subject susceptible to or afflicted with long COVID, the method comprising administering to the subject a therapeutically-effective combination of valacyclovir and celecoxib, wherein the amount of valacyclovir is administered in a total daily dose range from about 750 mg to about 2000 mg, and wherein the amount of celecoxib is administered in a total daily dose range from about 200 mg to about 800 mg, wherein the amount of nirmatrelvir is administered in a total daily dose range from about 100 mg to about 900 mg, which may be delivered once a day or twice daily, and wherein the amount of ritonavir is administered in a total daily dose range from about 100 mg to about 400 mg, which may be delivered once a day or twice daily, and wherein the combination administered produces no substantial adverse event.

In one embodiment, there is provided a method to treat a subject susceptible to or afflicted with PAIS, the method comprising administering to the subject a therapeutically-effective combination of famciclovir and celecoxib, wherein the amount of famciclovir is administered in a total daily dose range from about 250 mg to about 1000 mg, and wherein the amount of celecoxib is administered in a total daily dose range from about 200 mg to about 800 mg, wherein the amount of nirmatrelvir is administered in a total daily dose range from about 100 mg to about 900 mg, which may be delivered once a day or twice daily, and wherein the amount of ritonavir is administered in a total daily dose range from about 100 mg to about 400 mg, which may be delivered once a day or twice daily, and wherein the combination administered produces no substantial adverse event.

In one embodiment, there is provided a method to treat a subject susceptible to or afflicted with PAIS, the method comprising administering to the subject a therapeutically-effective combination of valacyclovir and celecoxib, wherein the amount of valacyclovir is administered in a total daily dose range from about 750 mg to about 2000 mg, and wherein the amount of celecoxib is administered in a total daily dose range from about 200 mg to about 800 mg, wherein the amount of nirmatrelvir is administered in a total daily dose range from about 100 mg to about 900 mg, which may be delivered once a day or twice daily, and wherein the amount of ritonavir is administered in a total daily dose range from about 100 mg to about 400 mg, which may be delivered once a day or twice daily, and wherein the combination administered produces no substantial adverse event.

In one embodiment, there is provided a method to treat symptoms of orthostatic intolerance in a subject suffering from PAIS, the method comprising administering to the subject a therapeutically-effective combination of valacyclovir and celecoxib, wherein the amount of valacyclovir is administered in a total daily dose range from about 750 mg to about 2000 mg, and wherein the amount of celecoxib is administered in a total daily dose range from about 200 mg to about 800 mg, wherein the amount of nirmatrelvir is administered in a total daily dose range from about 100 mg to about 900 mg, which may be delivered once a day or twice daily, and wherein the amount of ritonavir is administered in a total daily dose range from about 100 mg to about 400 mg, which may be delivered once a day or twice daily, and wherein the combination administered produces no substantial adverse event.

In a further embodiment, the method comprises an amount of each of the antiviral compound, the COX-2 inhibitor, and the one or more additional active ingredients (such as nirmatrelvir and ritonavir) present in a synergistically effective amount for treating long-COVID, PAIS, and/or orthostatic intolerance.

In a further embodiment, the one or more additional active ingredients (e.g., a combination of nirmatrelvir and ritonavir, molnupiravir, BCG vaccine, and/or ivermectin) is administered for an initial period of time in combination with the COX-2 inhibitor and the antiviral compound and treatment with the combination of the COX-2 inhibitor and the antiviral compound continues in the absence of the combination of nirmatrelvir, and ritonavir. For example, the one or more additional active ingredients may be administered to a subject during the first 5 to 30 days following identification of long-COVID, PAIS, or orthostatic intolerance. In particular, nirmatrelvir, and ritonavir may be administered for about 5 to 30 days following identification and/or diagnosis of long-COVID, PAIS, or orthostatic intolerance. The COX-2 inhibitor and the antiviral compound are administered during the timeframe of the one or more additional active ingredients (e.g., nirmatrelvir-ritonavir combination) treatment and continues for a period of time following cessation of nirmatrelvir-ritonavir administration (e.g., for about 1 additional week, about 2 additional weeks, about 3 additional weeks, about 1 month, about 2 months, about 3 months, about 4 months, about 5 months, about 6 months, about 7 months, about 8 months, about 9 months, about 10 months, about 11 months, about 12 months, about 2 years, about 3 years, or indefinitely).

### E. Subjects

Suitable subjects to be treated according to the present invention include mammalian subjects. Mammals according to the present invention include, but are not limited to, human, canine, feline, bovine, caprine, equine, ovine, porcine, rodents, lagomorphs, primates, and the like, and encompass mammals in utero. Subjects may be of either gender and at any stage of development. In a particular embodiment, the subject is a human.

### F. Combinations and Combination Therapy

The compounds of the present treatment, an antiviral compound, a COX-2 inhibitor, nirmatrelvir, and ritonavir, can be used as described herein or in combination with other pharmaceutically active compounds, to treat conditions such as those previously described above. The compounds of the present treatment, an antiviral compound, a COX-2 inhibitor, nirmatrelvir, and ritonavir, and other pharmaceutically active compound(s) can be administered simultaneously (either in the same dosage form or in separate dosage forms) or sequentially. Accordingly, in one embodiment, the present invention comprises methods for treating a condition by administering to the subject therapeutically-effective amounts of the compounds of the present invention, an antiviral compound, a COX-2 inhibitor, and one or more additional pharmaceutically active compounds.

In another embodiment, there is provided a pharmaceutical composition comprising the compounds of the present invention, an antiviral compound, a COX-2 inhibitor, and one or more additional pharmaceutically active compounds, and a pharmaceutically acceptable carrier.

In another embodiment, the one or more additional pharmaceutically active compounds are administered in any order or even simultaneously with the compounds of the present invention, an antiviral compound, a COX-2 inhibitor, nirmatrelvir, and ritonavir. If simultaneously, the multiple therapeutic agents are optionally provided in a single, unified form, or in multiple forms (by way of example only, either as a single pill or as two or more separate pills).

### EXAMPLES

The following examples are merely illustrative, and do not limit this disclosure in any way.

### Example 1: Human Clinical Trials

### Objective:

To explore the efficacy of the combination of celecoxib and famciclovir in the treatment of patients diagnosed with myalgic encephalomyelitis/chronic fatigue syndrome (ME/CFS).

### Study Design:

Thirty-three patients diagnosed with ME/CFS were treated with valacyclovir (average dose 2264 mg/d) and celecoxib (400 mg/d).

### Patient Population and Diagnostic Criteria:

Adult men and women with a documented diagnosis of ME/CFS were selected. Screening assessment included a medical and psychological history and physical examination.

### ME/CFS

Center for Disease Control Criteria for CFS diagnosis requires three criteria:
63) The individual has had severe chronic fatigue for 6 or more consecutive months that is not due to ongoing exertion or other medical conditions associated with fatigue
ii) The fatigue significantly interferes with daily activities and work
iii) The individual concurrently has 4 or more of the following 8 symptoms:
   (a) post-exertion malaise lasting more than 24 hours
   (b) unrefreshing sleep
   (c) significant impairment of short-term memory or concentration
   (d) muscle pain
   (e) pain in the joints without swelling or redness
   (f) headaches of a new type, pattern, or severity
   (g) tender lymph nodes in the neck or armpit
   (h) a sore throat that is frequent or recurring

These symptoms should have persisted or recurred during 6 or more consecutive months of illness and they cannot have first appeared before the fatigue. (Chronic fatigue syndrome: General information. Centers for Disease Control and Prevention.www.cdc.gov/cfs/general)

### Cognitive Dysfunction or Impairment

Cognitive dysfunction or impairment, also referred to as brain fog or mental fog, is the loss of intellectual functions (such as thinking, remembering, and reasoning) of sufficient severity to interfere with daily functioning. Patients with cognitive dysfunction have trouble with verbal recall, basic arithmetic, and concentration.

Patient improvement was quantified using the Mental Clutter Scale (Leavitt, et al. (2011) Psychological Reports 109, 445-452).

### Fatigue

Fatigue is a feeling of a loss of energy, a strong desire to rest and sleep, and/or a feeling of being constantly overtired, which can interfere with normal daily activities. Fatigue can be assessed by a survey of patients receiving a combination of an antiviral and a COX-2 inhibitor, wherein the patients report the presence and severity of fatigue. Any improvement in fatigue is assessed by asking the patient first if fatigue was improved, and if so, to report the degree of improvement of their overall fatigue during the course of treatment.

### Results:

The results, presented in the tables below, show the relative improvement in fatigue and cognitive function. The results are based on patients' self-reported improvement in their symptoms of fatigue and cognition. Table 1 shows the results of the clinical trial. Table 2 shows the average dose and average improvement.

TABLE 1
Clinically - Treated Patient Responses

| | Valacy- | | | | |
|---|---|---|---|---|---|
| | clovir | Celecoxib | length of | Reported % Improvement | |
| Patient | dose | dose | treatment | Fa- | Cogni- |
| ID | (mg/d) | (mg/d) | (wks) | tigue | tion |
| LS | 2000 | 400 | 144 | 25 | 50 |
| DT | 3000 | 400 | 5 | 100 | 100 |
| TB | 3000 | 400 | 5 | 60 | 60 |
| IS | 2000 | 400 | 104 | 50 | 70 |
| PW | 2000 | 400 | 126 | 85 | 75 |
| RB | 2375 | 400 | 64.57 | 50 | 75 |
| KK | 2250 | 400 | 138 | 85 | 80 |
| CM | 2063 | 400 | 102.43 | 100 | 100 |
| JB | 2077 | 400 | 115.86 | 100 | 50 |
| SM | 3000 | 400 | 63.29 | 75 | 0 |
| BT | 2250 | 400 | 45 | 40 | 60 |
| SM | 2065 | 400 | 9.71 | 80 | 50 |
| DC | 2100 | 400 | 31.43 | 85 | 75 |
| SH | 2000 | 400 | 117 | 70 | 70 |
| KZ | 2214 | 400 | 35.86 | 10 | 30 |
| CH MO | 2000 | 400 | 58.71 | 30 | 30 |
| TB | 2077 | 400 | 144.86 | 65 | 60 |
| DT | 2106 | 400 | 114.86 | 80 | 100 |
| TB | 2058 | 400 | 31.14 | 60 | 60 |
| KW | 2000 | 400 | 151.29 | 80 | 75 |
| CF | 2000 | 400 | 115.14 | 40 | 0 |
| MA | 2500 | 400 | 52 | 95 | 95 |
| RB | 2000 | 400 | 321.29 | 95 | 98.5 |
| LB | 3000 | 400 | 12 | 95 | 97 |
| JB | 2500 | 400 | 14.71 | 50 | 50 |
| KC | 3000 | 400 | 3.43 | 72.5 | 62.5 |
| MF | 2000 | 400 | 42 | 85 | 80 |
| RB | 2038 | 400 | 18 | 20 | 20 |
| NG | 2000 | 400 | 169.43 | 99 | 100 |
| KM | 2038 | 400 | 179.57 | 87.5 | 100 |
| MB | 2000 | 400 | 145.43 | 30 | 30 |
| KZ | 3000 | 400 | 2.71 | 30 | 30 |
| SS | 2000 | 400 | 132.43 | 50 | 50 |

TABLE 2
Average Doses and Improvement

| Average | Average | Average | | |
|---|---|---|---|---|
| Valacyclovir | Celecoxib | Length of | | |
| dose | dose | Treatment | Average % Improvement | |
| (mg/d) | (mg/d) | (wks) | Fatigue | Cognition |
| 2264 | 400 | 85 | 66.0 | 63.1 |

### Example 2: Human Clinical Trial Protocol for Treatment of Long Covid

OBJECTIVE: To explore the safety and efficacy of the combination of an antiviral drug (famciclovir or valacyclovir)+celecoxib vs. placebo in the treatment of long COVID.

### Study Design:

An open-labeled, single-center, 14-week study was designed to explore the safety and efficacy of valacyclovir plus celecoxib for the treatment of prolonged symptoms caused by COVID-19 in adult female patients. The treatment consisted of daily doses of valacyclovir and celecoxib. Treated patients were dosed with 1.5 g valacyclovir and 200 mg celecoxib on a BID basis for 14 weeks in addition to the standard of care for treating long COVID. A comparison cohort of matching patients on standard of care was concurrently enrolled in which the comparison cohort received only standard of care to treat long COVID. The standard of care was current care provided by the center providers unless the long COVID patient was not a center patient. In that case, the standard of care was their ongoing care by PCP or another long COVID clinic.

Female patients were enrolled according to the inclusion criteria and assigned to treatment with either combination therapy (valcyclovir/celecoxib combination in addition to standard of care) (n=22) or placebo (standard of care alone) (n=17).

Qualified patients suffered primary long COVID (defined as experiencing one or more of fatigue, ongoing muscle weakness, reduced lung function, and below normal results in a 6-minute walking test [See Kuehn, JAMA, 325 (11) (2021)], and an absence of other infections or other conditions that could compromise the interpretation of study results.

Patients underwent initial screening procedures, after which they proceeded with the washout of excluded medications, if required. Patients dependent upon opioids or narcotics for pain control were not enrolled in the study.

Due to the celecoxib component, patients in the treatment group discontinued regular use of all other non-steroidal anti-inflammatory drugs (NSAIDs) at the time of randomization. Acetaminophen was utilized throughout the study at doses not to exceed 3250 mg per day. Patients continued low-dose aspirin for cardioprotection (<325 mg/day), triptans and ergotamines for migraine, and dopaminergic agents for restless leg syndrome, as well as muscle relaxants, sleeping aids and benzodiazepines (assuming no evidence of abuse or dependency).

Metabolic profiles of each patient's concomitant medications were assessed to ensure that there is no risk of significant drug-drug interactions with either drug. For drugs that are metabolized by CYP2C9, the concomitant use of fluconazole—a potent CYP2C9 inhibitor—was avoided.

After ensuring that all entry criteria have been satisfied and washout successfully completed, patients returned for baseline assessments and randomization. The day of the baseline assessments will be referred to as Day 0; patients initiated study drug either with the evening dose on Day 0 or the morning dose on the following day (Day 1), continuing with BID treatment for the duration of the study.

### Inclusion Criteria:

1. Willing and able to read, understand, and sign the informed consent.
2. Female, 18-65 years of age, inclusive.
3. Each female patient must have a negative urine pregnancy test at Screening and Baseline unless she is post-menopausal.
4. Females of child-bearing potential must be willing to utilize an effective birth control method for the duration of their study participation.
5. Diagnosis of primary long COVID.
6. A urine drug screen performed at the Screening Visit must be negative for drugs of abuse such as methamphetamine, cocaine, phencyclidine (PCP), and non-disclosed amphetamines and opioids/opiates.
7. Qualified patients with mild to moderate depression should be clinically stable for three months, without risk of suicidal ideation or behavior. The dose of allowed antidepressants should have been stable for at least three months prior to screening.
8. In the opinion of the Investigator, the patient is willing and able to comply with all protocol-specified requirements.

### Exclusion Criteria:

1. Breastfeeding or pregnant.
2. Diagnosed with failed back syndrome, infectious arthritis, rheumatoid arthritis, systemic lupus erythematosus, or other systemic auto-immune diseases.
3. In the opinion of the Investigator, any clinically significant, uncontrolled or unstable medical, psychiatric or surgical condition that could affect the patient's ability to participate in the study or potentially compromise his/her well-being while enrolled in the study.
4. History of significant adverse reaction or allergy to study drugs.
5. History of suicide attempt or other suicidal behavior in the previous two years.
6. Any anticipated need for surgery that might confound results or interfere with the patient's ability to comply with the protocol.
7. Symptomatic and/or otherwise clinically significant cardiac disease.
8. Acute non-COVID systemic infection (e.g., HIV, hepatitis) or other active viral or bacterial infection during Screening or at the Baseline visit.
9. Currently receiving chronic systemic corticosteroids (>5 mg prednisone daily, or equivalent).
10. Uncontrolled sleep apnea. Patients successfully treated with CPAP or other devices are eligible.
11. Use of chronic nucleoside analog antiviral suppression therapy within one month of the Screening Visit, or requiring on average more than one acute treatment course every two months.
12. Current use of celecoxib in combination with valacyclovir or famciclovir.
13. The patient has undergone a malabsorptive weight loss procedure (e.g., Roux-en-Y or other bypass procedure).
14. Severe IBS-C or colonic inertia as evidenced by seven or more days between bowel movements.
15. In the opinion of the Investigator, evidence of clinically significant laboratory abnormality(ies) based on the results of the screening laboratory assessments and/or medical history.

### Study Drugs:

The study drug was commercially available generic valacyclovir tablets and celecoxib capsules. At Baseline, a 45-day supply of study drug was dispensed to patients in the treatment arm, and a 30-day supply was dispensed at Weeks 6 and 10. Each dose of study drug contained one 200 mg celecoxib capsule and 1.5 grams of valacyclovir. Patients were instructed to take each dose twice daily (BID).

### Results:

### Efficacy Measures:

The primary outcome measure was change from baseline to Week 14 in the PROMIS fatigue 8a T-score

Secondary measures will include:
change from baseline to Week 6, 10, and 14 in the PROMIS fatigue 8a T-score;
change from baseline to Weeks 6, 12 and 14 in the self-reported NRS fatigue score;
change from baseline to Weeks 6, 10, and 14 in the self-reported average pain intensity score;
patient global status as measured on two PGIC scales at Weeks 6, 10, and 14;
change from baseline to Weeks 6, 10, and 14 in the HADS Anxiety domain and the HADS Depression domain; and
Change from baseline to Weeks 6, 10, and 14 in orthostatic intolerance testing.

### Safety Measures:

Safety measures included vital signs (sitting blood pressure and heart rate, oral temperature, weight), adverse events and clinical laboratory assessments.

### Statistical Analyses:

The primary efficacy assessment for the determination of therapeutic efficacy was the change from baseline in the symptoms of long COVID. Change from baseline will be determined by comparing the baseline in the symptoms of long COVID to that determined at Weeks 6, 10, and 14.

The mean change from baseline in the combination drug treatment groups was compared to that determined for the placebo treatment group over 16 weeks of treatment using a mixed model repeated measures (MMRM). The null hypothesis was that there is no difference between treatment groups in terms of the mean change from baseline. Rejection of this hypothesis indicated efficacy of the combination therapy.

### Results:

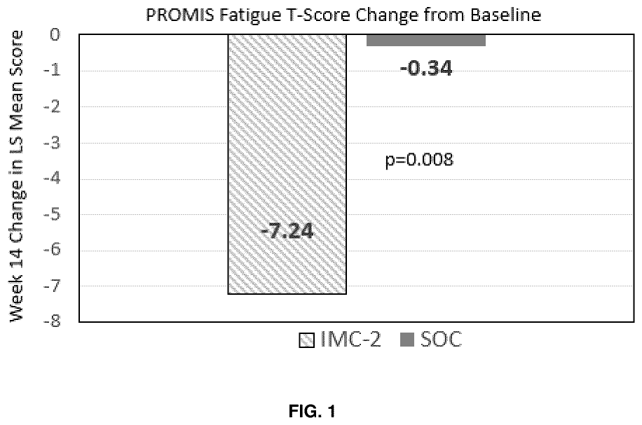

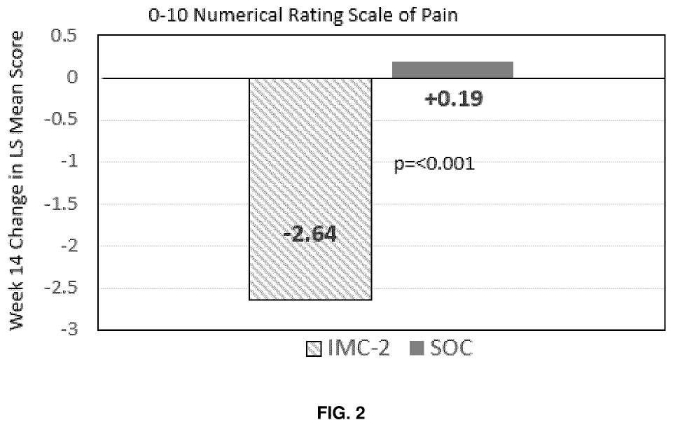

Tables 3 and 4 show the PROMIS fatigue score and NRS Fatigue score, respectively, in the subjects treated with the combination of 1.5 g valacyclovir and 200 mg celecoxib (represented as “IMC-2”). The “within group p value” column shows the statistical significance of the change in score as compared to baseline. The “comparative p value” column represents the statistical significance of the score between the treatment group (e.g., those patients that received the valacyclovir and celecoxib combination) with the standard of care (SOC) group (e.g., those patients that did not receive the valacyclovir and celecoxib combination). Addition of the valacyclovir/celecoxib combination to standard of care resulted in a statistically significant improvement in fatigue (as measured by both PROMIS and NRS scales) as compared to baseline. In contrast, subjects receiving only standard of care did not show a statistically significant improvement in fatigue as compared to baseline. Further, the results show a statistically significant improvement in most fatigue scores between the treatment group and control group. FIG. 1 shows the PROMIS fatigue T score change from baseline for both the combination and SOC control at Week 14. FIG. 2 shows the NRS fatigue score change from baseline for both the combination and SOC control at Week 14.

TABLE 3

| | | IMC-2 | | SOC |
|---|---|---|---|---|
| PROMIS | | change | | change |
| Fatigue | | from | | form |
| Score | IMC-2 score | baseline | SOC score | baseline |
| Baseline | 66.29 (n = 21) | | 66.53 (n = 17) | |
| Week 6 LS | 61.48* (n = 21) | −4.89 | 65.48 (n = 17) | −0.90*^{∧} |
| mean score | | | | |
| Week 10 LS | 60.26* (n = 21) | −6.12 | No score taken | |
| mean score | | | | |
| Week 14 LS | 59.14* (n = 20) | −7.24 | 66.04 (n = 16) | −0.34*^{∧} |
| mean score | | | | |

*statistical significance of p < 0.05 as compared baseline value of same treatment group
^{∧}statistical significance of p < 0.05 between treatment group (IMC-2) and control group (SOC).

TABLE 4

| | | IMC-2 | | SOC |
|---|---|---|---|---|
| NRS Fatigue | | change | | change |
| Scale (0-10, 0 | | from | | form |
| is no fatigue) | IMC-2 score | baseline | SOC score | baseline |
| Baseline | 6.75 (n = 20) | | 6.94 (n = 17) | |
| Week 6 LS | 5.11* (n = 21) | −1.80 | 5.92* (n = 17) | −0.99 |
| mean score | | | | |
| Week 10 LS | 4.69* (n = 20) | −2.22 | 6.45 (n = 17) | −0.46^{∧} |
| mean score | | | | |
| Week 14 LS | 4.45* (n = 20) | −2.64 | 7.10 (n = 16) | +0.19^{∧} |
| mean score | | | | |

*statistical significance of p < 0.05 as compared baseline value of same treatment group
^{∧}statistical significance of p < 0.05 between treatment group (IMC-2) and control group (SOC).

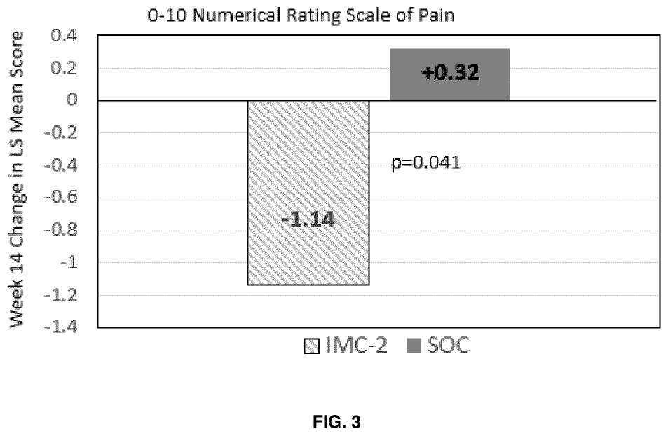

Table 5 shows the NRS pain scale in the subjects treated with the combination of 1.5 g valacyclovir and 200 mg celecoxib (represented as “IMC-2”). Addition of the valacyclovir/celecoxib combination to standard of care resulted in a statistically significant improvement in pain as compared to control subjects. FIG. 3 shows the NRS pain scale change from baseline for both the combination and SOC control at Week 14.

TABLE 5

| | | IMC-2 | | SOC |
|---|---|---|---|---|
| NRS Pain | | change | | change |
| Scale (0-10, 0 | | from | | form |
| is no pain) | IMC-2 score | baseline | SOC score | baseline |
| Baseline | 3.76 (n = 20) | | 4.56 (n = 17) | |
| Week 6 LS | 4.11* (n = 21) | −0.09 | 4.86 (n = 17) | +0.66 |
| mean score | | | | |
| Week 10 LS | 3.52* (n = 20) | −0.68 | 5.06 (n = 17) | +0.86^{∧} |
| mean score | | | | |
| Week 14 LS | 3.06* (n = 20) | −1.14 | 4.51 (n = 16) | +0.31^{∧} |
| mean score | | | | |

*statistical significance of p < 0.05 as compared baseline value of same treatment group
^{∧}statistical significance of p < 0.05 between treatment group (IMC-2) and control group (SOC).

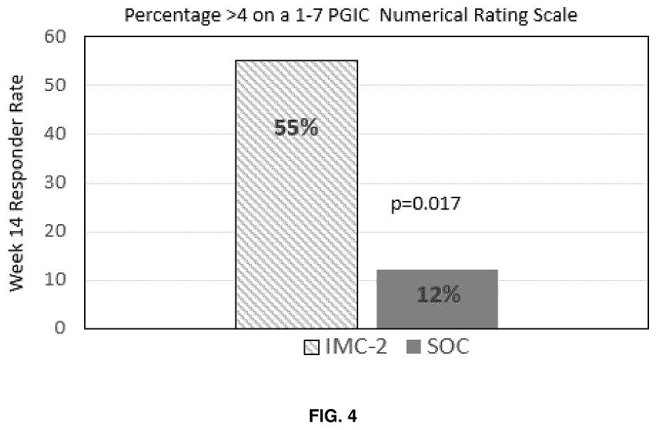

Tables 6 and 7 show the Patient Global Impression Change (PGIC) 1-7 scale and PGIC Improved 0-10 scale scores in the subjects treated with the combination of 1.5 g valacyclovir and 200 mg celecoxib (represented as “IMC-2”). Addition of the valacyclovir/celecoxib combination to standard of care resulted in a statistically significant improvement in PGIC and PGIC Improved as compared to control subjects. FIG. 4 shows the PGIC responder rate at Week 14. Tables 8 and 9 show the percentage of subjects reporting a “good result” according to PGIC and PGIC Improved. Here, a “good result” for the PGIC 1-7 scale refers to subjects that achieved a score of 5, 6, or 7 on the PGIC scale relative to their initial scores. A “good result” for the PGIC 0-10 scale refers to subject that achieves a score below 4 relative to their initial scores.

TABLE 6

| | | | Difference |
|---|---|---|---|
| | | | between |
| PGIC (1-7, 7 | | | IMC-2 and |
| is best) | IMC-2 score | SOC score | SOC groups |
| Week 6 LS mean score | 3.67 (n = 21) | 2.29 (n = 17) | 1.37^{∧} |
| Week 10 LS mean score | 4.02 (n = 20) | Not measured | |
| Week 14 LS mean score | 3.93 (n = 20) | 2.53 (n = 17) | +1.4^{∧} |

^{∧}statistical significance of p < 0.05 between treatment group (IMC-2) and control group (SOC).

TABLE 7

| | | | Difference |
|---|---|---|---|
| | | | between |
| PGIC Improved (0-10, 0 | | | IMC-2 and |
| is best) | IMC-2 score | SOC score | SOC groups |
| Week 6 LS mean score | 4.00 (n = 21) | 5.75 (n = 17) | −1.75^{∧} |
| Week 10 LS mean score | 3.48 (n = 20) | Not measured | |
| Week 14 LS mean score | 3.59 (n = 20) | 5.13 (n = 17) | −1.54^{∧} |

^{∧}statistical significance of p < 0.05 between treatment group (IMC-2) and control group (SOC).

TABLE 8

| | IMC-2 # of | | SOC # | |
|---|---|---|---|---|
| | “good | IMC-2 “good | of “good | SOC “good |
| PGIC | results” | result” rate | results” | result” rate |
| Week 6 | 10/21 | 45% | 3/17 | 18% |
| Week 10 | 10/21 | 48% | Not measured | |
| Week 14 | 11/20 | 55% | 2/11 | 12%^{#} |

^{#}statistical significance of Fisher Exact Score <0.05 between treatment group (IMC-2) and control group (SOC).

TABLE 9

| | IMC-2 # of | | SOC # | |
|---|---|---|---|---|
| PGIC | “good | IMC-2 “good | of “good | SOC “good |
| Improved | results” | result” rate | results” | result” rate |
| Week 6 | 9/21 | 43% | 0/7 | 0%^{#} |
| Week 10 | 9/20 | 45% | Not measured | |
| Week 14 | 16/20 | 55% | 1/11 | 6%^{#} |

^{#}statistical significance of Fisher Exact Score <0.05 between treatment group (IMC-2) and control group (SOC).

Tables 10 and 11 show the Hospital Anxiety and Depression Scale (HADS) depression and anxiety scores, respectively, in the subjects treated with the combination of 1.5 g valacyclovir and 200 mg celecoxib (represented as “IMC-2”). Addition of the valacyclovir/celecoxib combination to standard of care resulted in a statistically significant improvement in HADS depression score at Week 6 and in HADS Anxiety score at Week 14 as compared to control subjects.

TABLE 10

| | | IMC-2 | | SOC |
|---|---|---|---|---|
| | | change | | change |
| HADS | | from | | form |
| Depression | IMC-2 score | baseline | SOC score | baseline |
| Baseline | 8.90 (n = 21) | | 7.53 (n = 17) | |
| Week 6 LS | 5.75 (n = 21) | −2.64 | 8.84 (n = 17) | +0.45^{∧} |
| mean score | | | | |
| Week 10 LS | 5.44 (n = 21) | −2.95 | Not measured | |
| mean score | | | | |
| Week 14 LS | 5.85 (n = 20) | −2.54 | 7.78 (n = 16) | −0.61 |
| mean score | | | | |

^{∧}statistical significance of p<0.05 between treatment group (IMC-2) and control group (SOC).

TABLE 11

| | | IMC-2 | | SOC |
|---|---|---|---|---|
| | | change | | change |
| | | from | | form |
| HADS Anxiety | IMC-2 score | baseline | SOC score | baseline |
| Baseline | 8.14 (n = 21) | | 6.47 (n = 17) | |
| Week 6 LS | 7.08 (n = 21) | −0.45 | 7.24 (n = 17) | −0.29 |
| mean score | | | | |
| Week 10 LS | 5.93 (n = 21) | −1.60 | Not measured | |
| mean score | | | | |
| Week 14 LS | 2.35 (n = 20) | −1.27 | 8.30 (n = 16) | +0.77^{∧} |
| mean score | | | | |

^{∧}statistical significance of p<0.05 between treatment group (IMC-2) and control group (SOC).

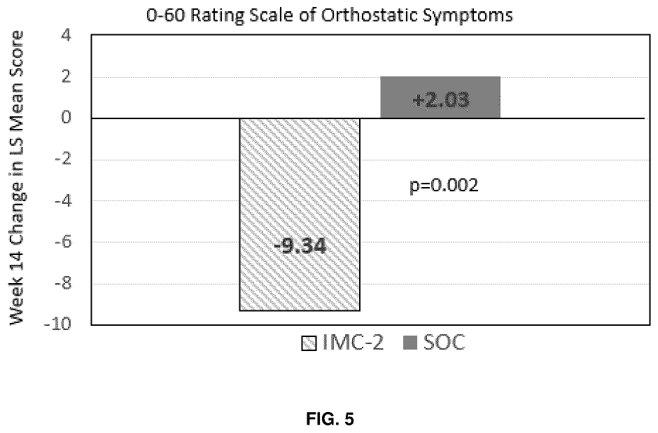

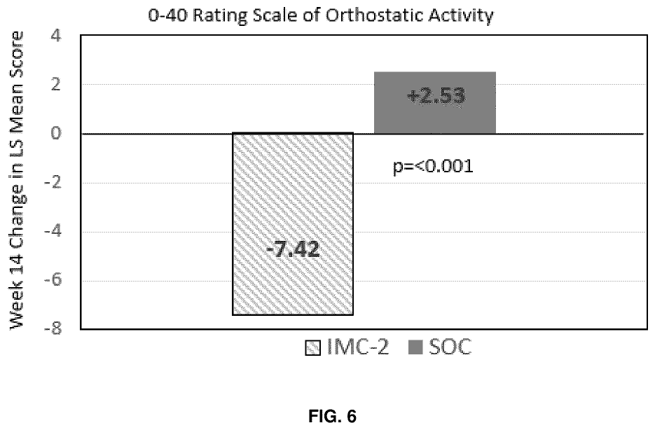

Tables 12 and 13 show the Orthostatic Intolerance Symptoms Assessment scale (OISA, OISAS) and Orthostatic Intolerance Daily Activity Impact Scale (OIDAS), respectively, in the subjects treated with the combination of 1.5 g valacyclovir and 200 mg celecoxib (represented as “IMC-2”). Addition of the valacyclovir/celecoxib combination to standard of care resulted in a statistically significant improvement in intolerance symptoms (as measured by OISA, OISAS) and daily activity (as measured by OIDAS) as compared to baseline. In contrast, subjects receiving only standard of care did not show a statistically significant improvement in either OISA, OISAS or OIDAS compared to baseline. Further, the results show a statistically significant improvement in OISA, OISAS and OIDAS scores between the treatment group and control group. FIG. 5 shows the OISA, OISAS change from baseline for both the combination and SOC control at Week 14. FIG. 6 shows the OIDAS change from baseline for both the combination and SOC control at Week 14.

TABLE 12

| | | IMC-2 | | SOC |
|---|---|---|---|---|
| | | change | | change |
| | | from | | form |
| OISA, OISAS | IMC-2 score | baseline | SOC score | baseline |
| Baseline | 35.76 (n = 21) | | 33.88 (n = 17) | |
| Week 6 LS | 27.78* (n = 21) | −7.76 | 36.62 (n = 17) | +1.94^{∧} |
| mean score | | | | |
| Week 10 LS | 27.82* (n = 20) | −7.54 | Not measured | |
| mean score | | | | |
| Week 14 LS | 26.02* (n = 20) | −9.34 | 37.39 (n = 16) | +2.03^{∧} |
| mean score | | | | |

*statistical significance of p<0.05 as compared baseline value of same treatment group
^{∧}statistical significance of p<0.05 between treatment group (IMC-2) and control group (SOC).

TABLE 13

| | | IMC-2 | | SOC |
|---|---|---|---|---|
| | | change | | change |
| | | from | | form |
| OIDAS | IMC-2 score | baseline | SOC score | baseline |
| Baseline | 22.76 (n = 21) | | 25.06 (n = 17) | |
| Week 6 LS | 17.85* (n = 21) | −5.84* | 25.21 (n = 17) | +1.52^{∧} |
| mean score | | | | |
| Week 10 LS | 17.55 (n = 20) | −6.14 | Not measured | |
| mean score | | | | |
| Week 14 LS | 16.27* (n = 19) | −7.42* | 26.04 (n = 17) | +2.53^{∧} |
| mean score | | | | |

*statistical significance of p < 0.05 as compared baseline value of same treatment group
^{∧}statistical significance of p < 0.05 between treatment group (IMC-2) and control group (SOC).

The results shown in Tables 3-11 and FIGS. 1-5 demonstrate that the combination of valacyclovir and celecoxib provides a statistically significant improvement over standard of care for long-COVID as measured by:
PROMIS fatigue at Weeks 6 and 14;
NRS fatigue scale at Weeks 10 and 14;
Global PGIC 1-7 scale continuous analysis at Weeks 6 and 14;
Global PGIC 1-7 scale responder analysis at Weeks 6 and 14;
Global PGIC 0-10 scale continuous analysis at Weeks 6 and 14;
Global PGIC 0-10 responder analysis at Weeks 6 and 14;
Orthostatic intolerance symptoms assessment (OISA) at Weeks 6 and 14;
Orthostatic intolerance daily activity impact (OIDAS) at Weeks 6 and 14;
NRS pain scale at Weeks 10 and 14; and
HADS anxiety and depression scale.

### Example 3: Treatment of Long-COVID with Valacyclovir, Celecoxib, and Paxlovid™

### Objective:

To explore the efficacy of the combination of celecoxib, valacyclovir, nirmatrelvir, and ritonavir in the treatment long-COVID.

### Study Design:

Inventors sought an improvement for treatment for long COVID in patients receiving valacyclovir and celecoxib. In an initial, preliminary trial, inventors provided subjects suffering long COVID with a 15-day course of PAXLOVID™ (nirmatrelvir and ritonavir) with concurrent treatment of 750 mg valacyclovir and 200 mg celecoxib. The subjects in the initial trial had few complaints and showed a dramatic recovery to long COVID as compared to those subjects that did not receive the initial 15-day course of nirmatrelvir and ritonavir.

Based on the success of the preliminary trial, the inventors began an open-label study to demonstrate the efficacy of valacyclovir and celecoxib to treat long COVID and synergy with the combination of nirmatrelvir and ritonavir. A total of 20 patients were enrolled in the open-label trial (N=12 for treatment group; N=11 for control group). Three subjects in the control group were subsequently treated with the combination of nirmatrelvir, and ritonavir plus valacyclovir and celecoxib and are included in both the control and treatment group data. Subjects in the treatment group received twice daily dosing of 300 mg nirmatrelvir and 100 mg ritonavir for fifteen days. Subjects in both the treatment and control groups received 750 mg valacyclovir dosed twice a day and 200 mg celecoxib, also dosed twice a day.

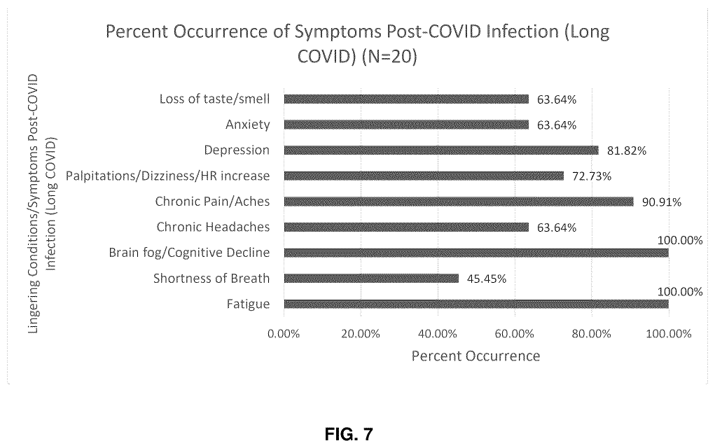

The primary endpoint was the change in PROMIS Fatigue T-Score from baseline. The subjects in the treatment group received a 15-day treatment of the combination of nirmatrelvir, and ritonavir plus valacyclovir and celecoxib during the acute phase of the COVID-19 infection followed by chronic treatment with valacyclovir and celecoxib. The subjects in the control group received valacyclovir and celecoxib throughout the entire trial but did not receive any nirmatrelvir-ritonavir combination. To be included in the trial, the subjects must be 18-75 years old, claim they are substantially less healthy after the SARS-COV-2 pandemic, and suffer from severe or moderately severe fatigue, moderately severe brain fog or palpitations (with dizziness and tachycardia) or both severe or moderately severe fatigue and moderately severe brain fog or palpitations. Subjects with long-COVID symptoms may or may not have tested positive for SARS-COV-2 infection or even known that they were infected. The subjects manifested classic long-COVID symptoms. FIG. 7 shows the percent occurrence of long-COVID symptoms of the subjects. Endpoint data was collected prospectively for IMC-2 (e.g., the control group) on month 4. Data was collected prospectively for the combination of nirmatrelvir, and ritonavir plus valacyclovir and celecoxib (e.g., the treatment group) at 1-month increments and the 4-month data was used for endpoint comparison. End Points were change of severity of fatigue, brain fog, and dysautonomia symptoms (0-100) NRS at 120 days.

### Results:

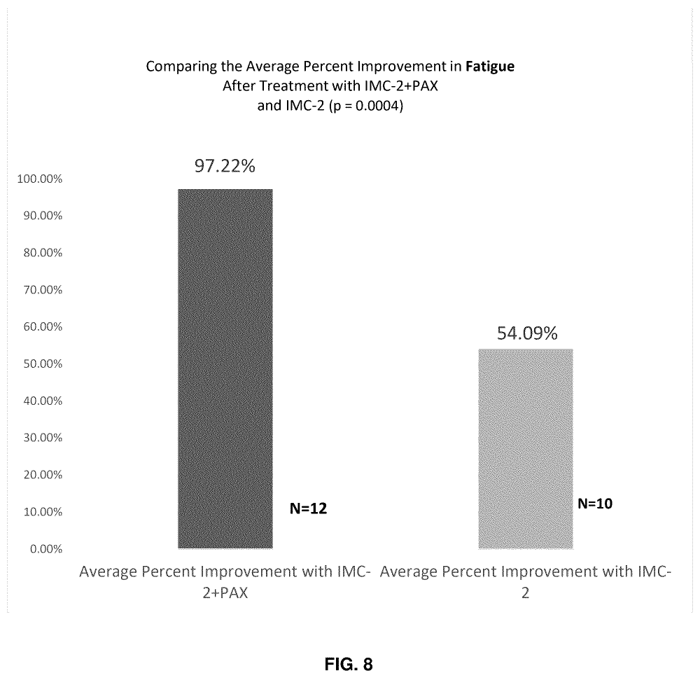

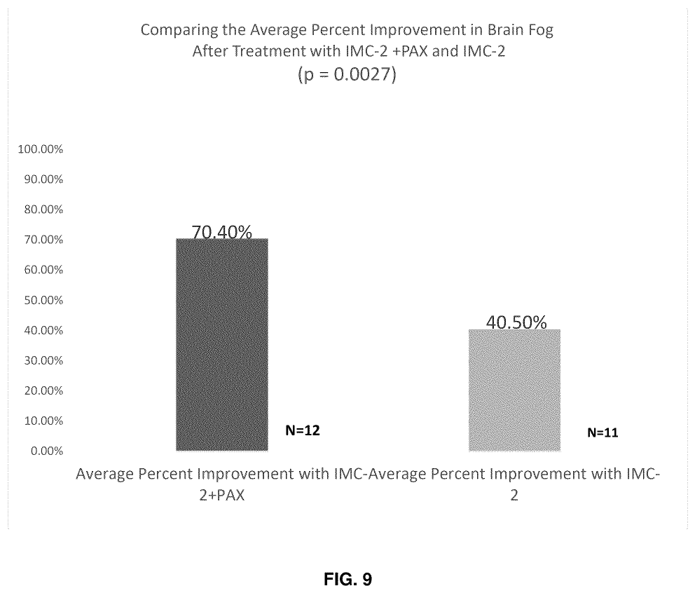

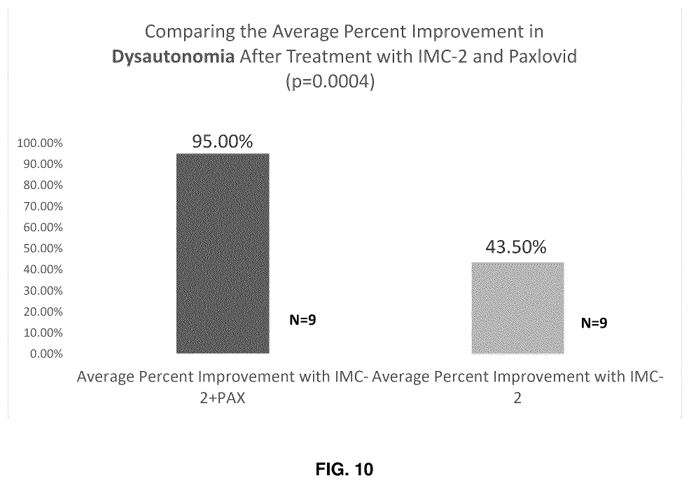

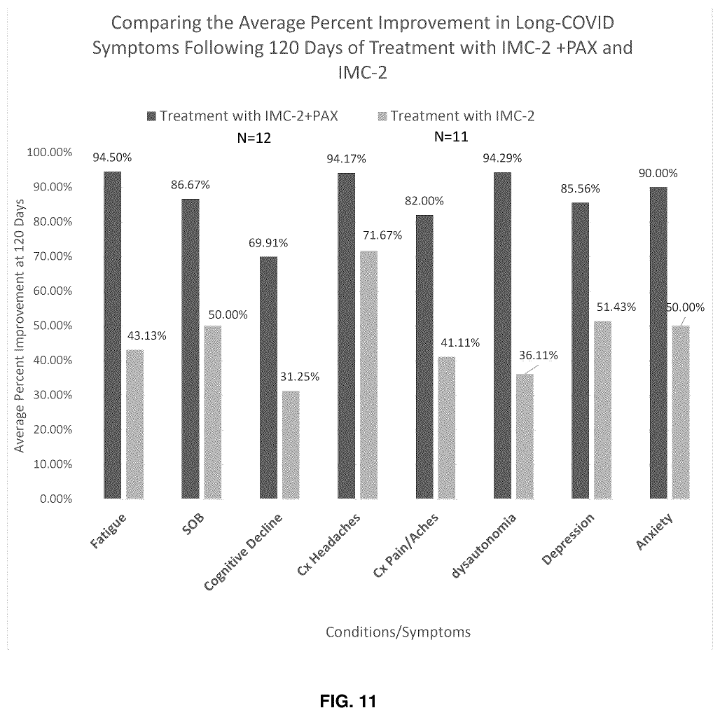

The addition of the nirmatrelvir-ritonavir combination to valacyclovir and celecoxib showed statistically significant synergy of the four drugs as compared to treatment with only valacyclovir and celecoxib. As noted above, other groups have attempted to treat long-COVID with the nirmatrelvir-ritonavir combination alone, but the study was abandoned early due to a lack of efficacy. FIG. 8 compares the average percent improvement in fatigue following treatment with the nirmatrelvir-ritonavir combination plus valacyclovir and celecoxib (represented as “IMC-2”) (97.22%) with treatment with valacyclovir and celecoxib alone (54.09%). FIG. 9 shows the average percent improvement in brain fog following treatment with the nirmatrelvir-ritonavir combination plus valacyclovir and celecoxib (represented as “IMC-2”) (70.40%) with treatment with valacyclovir and celecoxib alone (40.50%). FIG. 10 compares the average percent improvement in dysautonomia (e.g., palpitations, tachycardia, and dizziness) following treatment with the nirmatrelvir-ritonavir combination plus valacyclovir and celecoxib (represented as “IMC-2”) (95.00%) with treatment with only valacyclovir and celecoxib (43.50%). FIG. 11 compares the average improvement in long-COVID symptoms or conditions following treatment with the nirmatrelvir-ritonavir combination plus valacyclovir and celecoxib and as compared to treatment with only valacyclovir and celecoxib alone. Subjects receiving the nirmatrelvir-ritonavir combination plus valacyclovir and celecoxib (represented as “IMC-2”) show a consistent, statistically significant improvement (e.g., p<0.05) in all measured long-COVID symptoms as compared to the control group.

Of the subjects in the treatment group: 97.2% showed a reduction in all fatigue symptoms; 58.3% had complete (e.g., 100%) reduction in all fatigue symptoms; 83% with dysautonomia symptoms showed 100% resolution of their palpitations, tachycardia, and dizziness; 50% of patients with pain showed complete (e.g., 100%) resolution of pain; 50% of subjects with depression showed complete (e.g., 100%) resolution of depression; 50% of subjects with anxiety showed complete (e.g., 100%) resolution of anxiety; and all subjects with brain fog showed improvement, with an average improvement of 82%.

The results described above are consistent with the recent popular theory that long-COVID appears to awaken latent viruses, which become problematic. The combination of an antiviral compound (e.g., valacyclovir) and a COX-2 inhibitor (e.g., celecoxib) has significant long-term efficacy against the symptoms of long-COVID. The combination of nirmatrelvir and ritonavir (e.g., PAXLOVID™) has been shown to be ineffective against symptoms of long-COVID. The data shown above show a clear synergy when valacyclovir and celecoxib are given alongside a 15-day course of nirmatrelvir and ritonavir. The valacyclovir and celecoxib provide viral suppression following the completed nirmatrelvir/ritonavir course needed to prevent a reawakening of dormant herpesviruses, which results in a complete or near complete recovery of all long-COVID symptoms. Such results suggest a similar response when other anti-COVID treatments, such as molnupiravir (LAGEVRIO™), BCG vaccine, and/or ivermectin, are provided in combination with valacyclovir and celecoxib.

### Example 4: Treatment of Long-COVID with Valacyclovir, Celecoxib, and Paxlovid™

### Objective:

To further explore the efficacy of the combination of celecoxib, valacyclovir, nirmatrelvir, and ritonavir in the treatment of long-COVID.

### Study Design:

Inventors sought to further explore treatment for long COVID in patents receiving valacyclovir and celecoxib (IMC-2). Examples 3 and 4 are a part of a 2-parts study. Here, in a 120-day, open label, LC study, 24 male and female patients were treated. 12 were treated with IMC-2 and 15 patients were given continuous IMC-2 with 15-days of Paxlovid. 3 patients participated in both arms. A rating scale of 0 to 100% improvement of symptoms from baseline was used. Primary and secondary outcomes measures included a change in fatigue, brain fog, and dysautonomia. Long term data was similarly collected at the triple therapy 305-day average.

Patients had LC symptoms (fatigue, brain fog and dysautonomia) for at least 5 months (with or without a positive test for SARS COV-2). Symptoms were rated using a five-point (none, mild, moderate, moderately severe, & severe) grading scale at the time of enrollment. During or following the pandemic, all patients must claim that they were substantially less healthy. Secondly, all patients must have suffered from severe or moderately severe fatigue in addition to moderately severe brain fog and/or moderately severe dysautonomia.

Percentage improvement (0-100) from the nadir of symptoms were compared to those symptoms on the day of the visit. The primary endpoint was a change of severity of fatigue. The secondary endpoints were a change in brain fog and dysautonomia symptoms at 120 days. Fatigue questioning focused on a lack of energy and mental exhaustion. Brain fog questioning focused on forgetfulness, sluggish recall and trouble focusing. Dysautonomia questioning centered around tachycardia, dizziness, presyncope, syncope or palpitations. Statistical analysis utilized overlapping paired samples t-test.

### Results:

The primary endpoint was a change in fatigue, and the secondary endpoints included changes in brain fog and dysautonomia. Triple therapy resulted in a 92.0, 83.9, and 93.3% reduction of symptoms respectively, compared to 52.9, 43.3 and 41.7% reduction for these same symptoms with IMC-2 alone. (p-values 0.00023, 0.00051, and 0.00086)

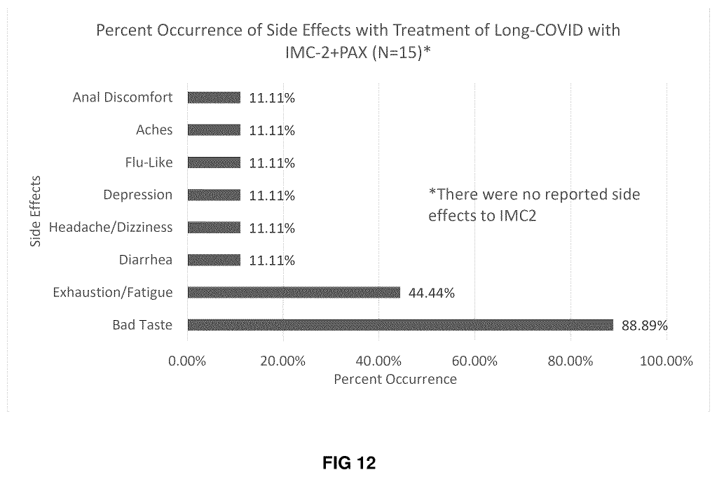

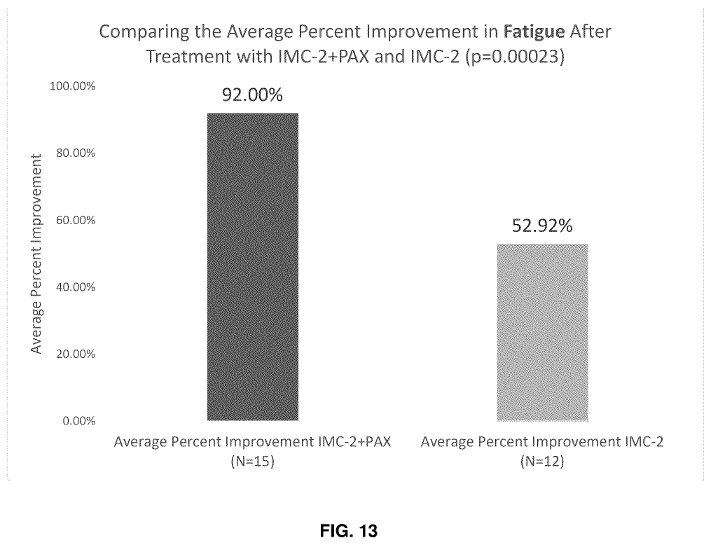

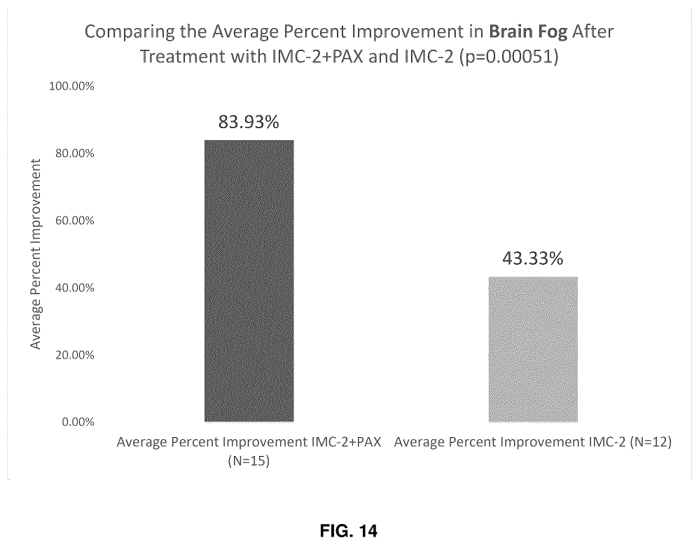

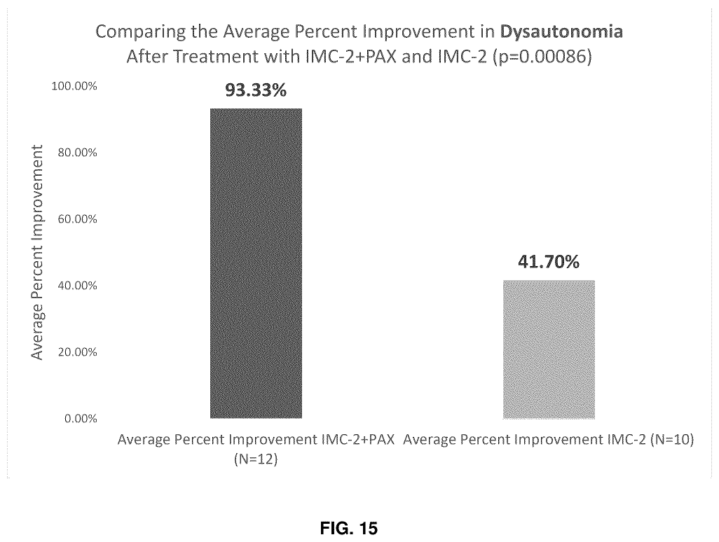

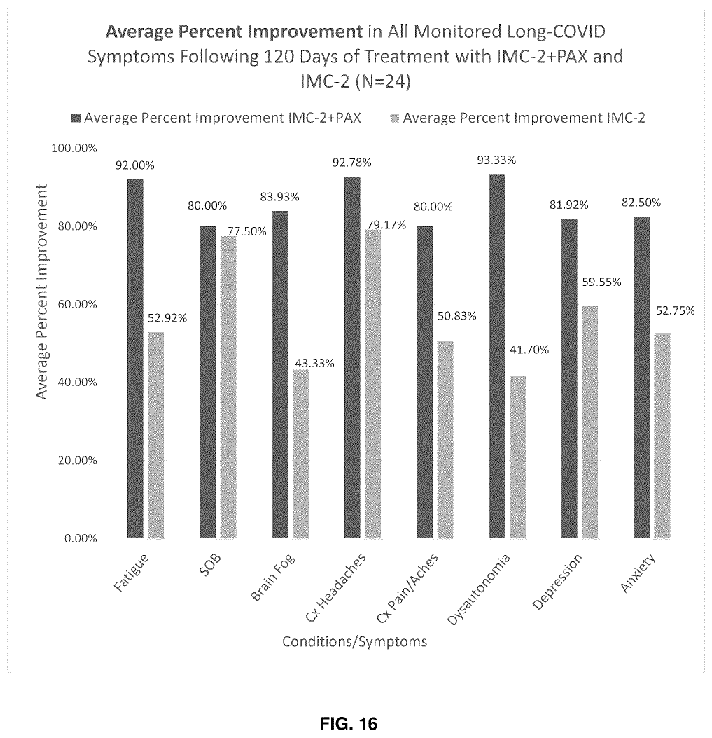

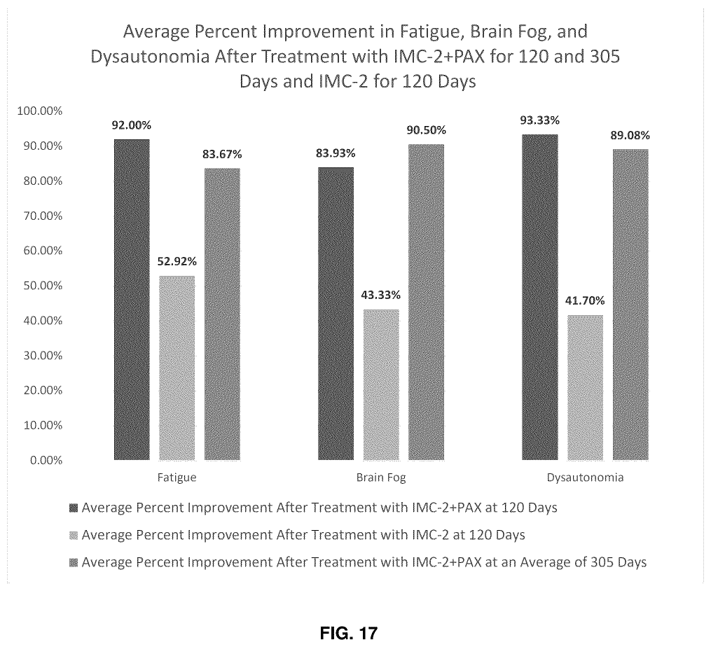

FIG. 12 shows that there were no reported side effects for the valacyclovir and celecoxib fixed dose combination (IMC2). FIG. 13 shows a 52.92% average reduction in all fatigue symptoms for the 12 IMC-2 only patients, while there was a 92.0% average reduction in all fatigue symptoms in the 15 triple therapy patients. FIG. 14 shows the reduction in severity of symptoms of the secondary endpoint, brain fog, was an average of 43.33% for the 12 patients who were treated with IMC-2 alone and an average 83.93% for the 15 patients who were treated with triple therapy. FIG. 15 shows the reduction in the secondary endpoint, dysautonomia symptoms, was an average of 41.7% for the 10 patients who took IMC-2 only, and an average 93.3% improvement for the 12 patients who took triple therapy. FIG. 16 shows the complete data summary for the 24 patients and 8 symptoms. FIG. 17 shows the data comparing the primary endpoint and secondary endpoints at 120-day versus the 305-day (avg.) long-term data demonstrate the durability of triple therapy.

Paxlovid has activity against SARS-COV-2. Nirmatrelvir, a protease inhibitor, inhibits viral replication by cleaving viral polyproteins involved in replication. Ritonavir, has no activity against SARS-COV-2, yet its role is to specifically inhibit the metabolism of nirmatrelvir by CYP3A (cytochrome P450-3A), thus boosting its potency. Paxlovid, due to its 3C-like proteases (cysteine), potentially has activity against over a dozen different viruses. It is believed that 15-days of Paxlovid should have activity for any residual SARS COV-2 in the brain, respiratory, GI tract or other sites. The addition of IMC-2 theoretically provided continuous daily viral suppression, thus reversing the viral reactivation of herpes viruses and potentially other viruses. This study took valacyclovir and celecoxib, a combination that was commonly prescribed for FM, IBS, and MECFS patients, and recognized that combining it with a third agent Paxlovid, could provide an additional layer of treatment. This triple therapy apparently avoids viral adaptation.

The results described above for the role of IMC-2 in the treatment of LC are consistent with the recent popular theories about the causes of LC. First, it appears that the immune system is altered or develops a form of exhaustion resulting in the awakening of latent viruses which wreak havoc. Second, an acute SARS COV-2 infection results in residual SARS COV-2 in multiple sites throughout the body, thus triggering an ongoing immune response. The role of Paxlovid is to eradicate these remaining SARS COV-2 halting a chronic reaction to the virus itself or there may also be an immune reset of sorts. It would seem that IMC-2 has significant long-term efficacy against the symptoms of Long COVID. Paxlovid by itself appears to lack efficacy against LC. The study shows synergy of IMC-2 and Paxlovid as evidenced by a near-complete resolution of LC symptoms at the 120-day and 305-day mark.

All mentioned documents are incorporated by reference as if herein written. When introducing elements of the present invention or the exemplary embodiment(s) thereof, the articles “a,” “an,” “the” and “said” are intended to mean that there are one or more of the elements. The terms “comprising,” “including” and “having” are intended to be inclusive and mean that there may be additional elements other than the listed elements. Although this invention has been described with respect to specific embodiments, the details of these embodiments are not to be construed as limitations.

1-51. (canceled)

52. A method to treat a subject susceptible to or afflicted with long-COVID, or a post-acute infection syndrome (PAIS) or symptoms of orthostatic intolerance in subjects with post-acute infection syndrome (PAIS), the method comprising:
administering to the subject a therapeutically effective amount of an antiviral compound, a therapeutically effective amount of a COX-2 inhibitor, a therapeutically effective amount of one or more additional active ingredients,
wherein the one or more additional active ingredients is selected from the group consisting of a combination of a therapeutically effective amount of nirmatrelvir and a therapeutically effective amount of ritonavir, a therapeutically effective amount of molnupiravir, a therapeutically effective amount of Bacilli Calmette-Guerin (BCG) vaccine, and a therapeutically effective amount of ivermectin.

53. The method of claim 52, wherein the antiviral compound is famciclovir or valacyclovir.

54. The method of claim 52, wherein the COX-2 inhibitor is celecoxib.

55. The method of claim 52, wherein the antiviral compound is famciclovir and the COX-2 inhibitor is celecoxib.

56. The method of claim 55, wherein the amount of famciclovir is administered in a total daily dose range from about 750 mg to about 1500 mg, and wherein the amount of celecoxib is administered in a total daily dose range from about 200 mg to about 800 mg.

57. The method of claim 52, wherein the antiviral compound is valacyclovir and the COX-2 inhibitor is celecoxib.

58. The method of claim 57, wherein the amount of valacyclovir is administered in a total daily dose range from about 750 mg to about 2000 mg, and wherein the amount of celecoxib is administered in a total daily dose range from about 200 mg to about 800 mg.

59. The method of claim 52, wherein the one or more additional active ingredients is the combination of nirmatrelvir and ritonavir, wherein the nirmatrelvir is administered twice daily in a dose range of about 50 mg to about 450 mg for each dose and the ritonavir is administered twice daily in a dose range of about 50 mg to about 200 mg.

60. The method of claim 52, wherein the combination of nirmatrelvir and ritonavir is administered daily for five to thirty days.

61. The method of claim 52, wherein the one or more additional active ingredients is molnupiravir and wherein the molnupiravir is administered twice daily in a dose of about 200 mg.

62. The method of claim 61, wherein the molnupiravir is administered daily for five to thirty days.

63. The method of claim 52, wherein the antiviral compound and the COX-2 inhibitor are administered daily for about 120 days.

64. The method of claim 52, wherein the amounts of the antiviral compound, the COX-2 inhibitor, and the one or more additional active ingredients are synergistically effective for treating long-COVID.

65. The method of claim 52, wherein the method comprises:
administering to the subject a therapeutically-effective combination of an antiviral component, a COX-2 inhibitor component, nirmatrelvir, and ritonavir, wherein the amount of the antiviral component is administered in a total daily dose range from about 250 mg to about 2000 mg, wherein the amount of the COX-2 inhibitor component is administered in a total daily dose range from about 15 mg to about 800 mg, wherein the amount of the nirmatrelvir is administered in a total daily dose range from about 100 mg to about 900 mg, and wherein the ritonavir is administered in a total daily dose range from about 100 mg to about 400 mg and wherein the combination administered produces no substantial adverse event.

67. A pharmaceutical composition comprising a therapeutically effective amount of an antiviral compound, a therapeutically effective amount of a COX-2 inhibitor, a therapeutically effective amount of one or more additional active ingredients,
wherein the one or more additional active ingredients is selected from the group consisting of a combination of a therapeutically effective amount of nirmatrelvir and a therapeutically effective amount of ritonavir, a therapeutically effective amount of molnupiravir, a therapeutically effective amount of Bacilli Calmette-Guerin (BCG) vaccine, and a therapeutically effective amount of ivermectin.

68. The pharmaceutical composition of claim 67, wherein the amounts of the antiviral compound, the COX-2 inhibitor, and the one or more additional active ingredients are synergistically effective for treating long-COVID, post-acute infection syndrome (PAIS), and/or orthostatic intolerance.

69. A kit comprising a therapeutically effective amount of valacyclovir or famciclovir in a first unit dosage form, a therapeutically effective amount of celecoxib in a second unit dosage form, and one or more additional active ingredients in additional dosage forms, wherein the first, second, and additional dosage forms are separately enclosed in one or more containers, and optionally comprising directions on how to use kit components,
wherein the one or more additional active ingredients is selected from the group consisting of a combination of a therapeutically effective amount of nirmatrelvir and a therapeutically effective amount of ritonavir, a therapeutically effective amount of molnupiravir, a therapeutically effective amount of Bacilli Calmette-Guerin (BCG) vaccine, and a therapeutically effective amount of ivermectin.

70. The kit of claim 69, wherein the amounts of the antiviral compound, the COX-2 inhibitor, the nirmatrelvir, and the ritonavir are synergistically effective for treating long-COVID, post-acute infection syndrome (PAIS), and/or orthostatic intolerance.
